(19) United States
(12) Patent Application Publication
(10) Pub. No.: US 2025/0336649 A1
(43) Pub. Date: Oct. 30, 2025

(54) APPARATUS FOR PLASMA SURFACE TREATMENT

(71) Applicant: PLASMAPP CO., LTD., Daejeon (KR)

(72) Inventor: You Bong LIM, Daejeon (KR)

(73) Assignee: PLASMAPP CO., LTD., Daejeon (KR)

(21) Appl. No.: 19/262,934

(22) Filed: Jul. 8, 2025

Related U.S. Application Data
(63) Continuation of application No. 18/692,172, filed on Mar. 14, 2024, now abandoned.

(30) Foreign Application Priority Data
Sep. 17, 2021 (KR) 10-2021-0124946
Sep. 17, 2021 (KR) 10-2021-0124952
Oct. 29, 2021 (KR) 10-2021-0146784
Dec. 24, 2021 (KR) 10-2021-0186977
Apr. 26, 2022 (KR) 10-2022-0051210
Apr. 29, 2022 (KR) 10-2022-0053605
Sep. 15, 2022 (KR) 10-2022-0116401

(51) Int. Cl. H01J 37/32; A61C 8/00; A61C 13/02

(52) U.S. Cl. CPC H01J 37/32513; A61C 8/0087; A61C 13/02; H01J 37/32568; H01J 37/32715; H01J 37/32834; H01J 37/32972; H01J 2237/335

(57) ABSTRACT

According to an exemplary embodiment of the present disclosure, an apparatus for plasma surface treatment is disclosed. The apparatus may include: a placement unit on which an object to be treated, a storage container for storing the object to be treated, or a gripping device for gripping the object to be treated is placed; an isolation unit that forms, by combining with the placement unit, an isolation space in which at least a part of an inside thereof is isolated from an external environment during an operation period of the apparatus, the isolation unit having at least one surface including a light-transmissive member; and a treatment unit that allows plasma surface treatment to be performed for the object to be treated by forming an electric field in the isolation space during the operation period of the apparatus.

### CROSS-REFERENCE TO RELATED APPLICATIONS

This application is a continuation of U.S. patent application Ser. No. 18/692,172, filed on Mar. 14, 2024, which has been allowed, which in turn claims priority to national stage entry of International PCT Application No. PCT/KR2022/013816 filed Sep. 15, 2022, which in turn claims priority to Korean Patent Application No. 10-2021-0124946 filed in the Korean Intellectual Property Office on Sep. 17, 2021; Korean Patent Application No. 10-2021-0124952 filed in the Korean Intellectual Property Office on Sep. 17, 2021; Korean Patent Application No. 10-2021-0146784 filed in the Korean Intellectual Property Office on Oct. 29, 2021; Korean Patent Application No. 10-2021-0186977 filed in the Korean Intellectual Property Office on Dec. 24, 2021; Korean Patent Application No. 10-2022-0051210 filed in the Korean Intellectual Property Office on Apr. 26, 2022; Korean Patent Application No. Oct. 10, 2022-0053605 filed in the Korean Intellectual Property Office on Apr. 29, 2022; and Korean Patent Application No. 10-2022-0116401 filed in the Korean Intellectual Property Office on Sep. 15, 2022. The entire contents of each of the foregoing applications are incorporated herein by reference.

### FIELD OF THE DISCLOSURE

The present disclosure relates to an apparatus that performs surface treatment for an object to be treated using plasma.

### BACKGROUND

An apparatus for plasma surface treatment modifies a surface of an object to be treated through plasma treatment.

Surface modification through the plasma treatment can modify the surface of the object to be treated from hydrophobicity to hydrophilicity. Such surface modification from hydrophobicity to hydrophilicity can be widely used in a medical field using artificial body parts such as implants.

As a specific example, the implant may include a fixture made of titanium or titanium alloy material, where a titanium surface may have hydrophobicity. Therefore, due to the hydrophobic nature of repelling moisture, after implantation of an implant, the synostosis of osseous tissues containing blood and protein may be delayed, or inflammation may occur due to the body's immune response. Since the apparatus for plasma surface treatment can modify the surface of the implant, which is an object to be treated, to be hydrophilic, the apparatus can suppress the delay in osseous tissue synostosis and the inflammatory response after implantation of the implant.

An implant can be distributed while sealed in packaging. If the implant is sealed in packaging after surface treatment, the surface energy may stabilize over time and the surface of the implant may change back to hydrophobicity. Therefore, the procedure should be performed before the surface of the implant is oxidized, which may shorten the storage period of the implant.

In this regard, Korea Patent Nos. 10-1439344 and 10-1693335 have been issued.

### SUMMARY OF THE DISCLOSURE

### Technical Problem

The present disclosure has been made in view of the above situations, and is intended to provide an apparatus for plasma surface treatment that can provide maximized user experience.

The technical problems addressed by the present disclosure are not limited to the problems described above, and other problems not described will be apparently understood by one skilled in the art from the following description.

### Technical Solution

In order to achieve the above object, according to some exemplary embodiments of the present disclosure, an apparatus for plasma surface treatment is disclosed. The apparatus includes: a placement unit on which at least one of an object to be treated, a storage container for storing the object to be treated, or a gripping device for gripping the object to be treated is placed; an isolation unit configured to form a sealed space in which an inside thereof is isolated from an external environment during an operation period of the apparatus, the isolation unit having at least one surface comprising a light-transmissive member; and a treatment unit configured to discharge plasma in the sealed space by forming electric field around the object during the operation period of the apparatus, and wherein light generated by the plasma is transmitted through the light-transmissive member.

In an exemplary embodiment, the isolation unit is configured such that the at least one surface allows transmission of visible light from the sealed space to an outside during at least one section of the operation period of the apparatus.

In an exemplary embodiment, the isolation unit is configured to allow transmission of visible light corresponding to a wavelength range of a region that reacts with plasma in the sealed space, during at least one section of the operation period of the apparatus.

In an exemplary embodiment, the region that reacts with the plasma corresponds to the object to be treated.

In an exemplary embodiment, the light-transmissive member is positioned at a position where the plasma is discharged.

In an exemplary embodiment, the isolation unit is configured to move from a first position where the isolation unit is accommodated inside an upper member defining a shape of at least a part of an upper part of the apparatus to a second position where the isolation unit forms, along with the placement unit, the sealed space, wherein at least a part of the isolation unit is accommodated in the upper member when the isolation unit is in the first position, wherein the isolation unit is taken out to outside of the upper member, while the isolation unit moves from the first position to second position, wherein one surface of the isolation unit comes into contact with one surface of the placement unit by relative movement of the isolation unit and the place unit so that an inside of the isolation unit can be formed with the sealed space, when the isolation unit is in the second position, and wherein the plasma discharged by the treatment unit occur inside of the isolation unit after forming the sealed space.

In an exemplary embodiment, the operation period of the apparatus is divided into a plurality of sections and the sections comprise: a first section during which the object to be treated and the placement unit are connected to each other, a second section during which the sealed space that blocks the object to be treated (IM) from the external environment is formed by the relative movement of the isolation unit and the placement unit, a third section during which plasma surface treatment is performed for the object to be treated by forming an electric field in the sealed space in a state in which the sealed space is formed, a fourth section during which the object to be treated is present inside the sealed space after the plasma surface treatment is completed, and a fifth section during which the sealed space is released by the relative movement of the isolation unit and the placement unit, the object to be treated is exposed to the external environment, and at least a part of the isolation unit is inserted into inside of the upper member.

In an exemplary embodiment, when the isolation unit is located at the first position, the isolation unit is not visible from an outside, and when the isolation unit is located at the second position, both the isolation unit and the object to be treated are visible from the outside, and wherein while the plasma surface treatment is performed for the object to be treated in a state in which the isolation unit is moved to the second position, a region of the object to be treated that reacts with plasma in the sealed space formed by the isolation unit changes dynamically.

In an exemplary embodiment, the object to be treated reacts with plasma sequentially along a longitudinal direction of the sealed space.

In an exemplary embodiment, an inner wall of the isolation unit is made of a dielectric barrier layer, and wherein when the isolation unit and the placement unit are in contact with each other and form the sealed space, power is applied to a first electrode which is provided in the placement unit and a second electrode which is provided in the isolation unit, so that plasma discharge occurs inside of the isolation unit forming the sealed space.

In an exemplary embodiment, a region of the object to be treated that reacts with the plasma in the sealed space is formed based on a structure of an exhaust unit connected to the sealed space, a dielectric structure inside the sealed space, and a structure of an electrode formed inside the sealed space.

In an exemplary embodiment, the object to be treated reacts with plasma sequentially in a direction of a first electrode located proximal to the placement unit, wherein at least a part of the first electrode is exposed to the inside of the sealed space formed by the isolation unit, and wherein the first electrode is electrically connected to the object to be treated present in the sealed space, a container in which the object to be treated is stored, or at least a part of the gripping device configured to grip the object to be treated.

In an exemplary embodiment, the isolation unit configured to form a sealed space in which an inside thereof is isolated from an external environment during an operation period of the apparatus.

In an exemplary embodiment, at least a part of the storage container comprises a light-transmissive member so that when the object to be treated is placed on the placement unit while stored in the storage container, the object to be treated is visible from the external environment during the operation period of the apparatus.

In an exemplary embodiment, at least a part of the gripping device comprises a light-transmissive member so that when the object to be treated is placed on the placement unit while gripped by the gripping device, the object to be treated is visible from the external environment during the operation period of the apparatus.

In an exemplary embodiment, at least a part of the gripping device comprises a surface at a position corresponding to a position on the gripping device where the object to be treated is present.

In an exemplary embodiment, the at least one surface of the isolation unit further comprises a conductive member, wherein the conductive member operates as at least a part of an electrode that forms the electric field inside the sealed space.

In an exemplary embodiment, at least a part of a surface of the sealed space comprises the light-transmissive member and at least a part of a surface of the sealed space comprises a conductive member.

In an exemplary embodiment, the isolation unit further comprises a conductive member disposed to occupy a partial region of the light-transmissive member along a region occupied by the light-transmissive member in the sealed space.

In an exemplary embodiment, the isolation unit further comprises a conductive member and wherein at least a part of regions occupied by the conductive member and the light-transmissive member overlaps.

### Advantageous Effects

According to some exemplary embodiments of the present disclosure for addressing the problems described above, an apparatus for plasma surface treatment by which user experience is maximized can be provided.

The effects obtainable in the present disclosure are not limited to the effects described above, and other effects not described will be apparently understood by one skilled in the art from the following description.

### BRIEF DESCRIPTION OF THE DRAWINGS

Various aspects will now be described with reference to the drawings. Here, similar reference numerals will be used to collectively refer to similar constitutional elements. In the following exemplary embodiments, for the sake of description, a plurality of specific details will be presented to provide comprehensive understanding of one or more aspects. However, it will be apparent that such aspect(s) may be implemented without such specific details.

FIG. 1 is a perspective view illustratively showing an apparatus for plasma surface treatment according to an exemplary embodiment of the present disclosure.

FIG. 2 is a front view illustratively showing the apparatus for plasma surface treatment according to the exemplary embodiment of the present disclosure.

FIG. 3 is a front view illustratively showing a state in which an object to be treated and a gripping device are placed on a placement unit of the apparatus for plasma surface treatment according to the exemplary embodiment of the present disclosure.

FIG. 4 is a conceptual diagram illustratively showing the apparatus for plasma surface treatment according to the exemplary embodiment of the present disclosure.

FIG. 5A is a perspective view illustratively showing the apparatus for plasma surface treatment according to the exemplary embodiment of the present disclosure.

FIG. 5B is a cross-sectional view illustratively showing the apparatus for plasma surface treatment according to the exemplary embodiment of the present disclosure.

FIG. 5C is a perspective view illustratively showing a state in which the object to be treated and the gripping device are placed on the placement unit of the apparatus for plasma surface treatment according to the exemplary embodiment of the present disclosure.

FIG. 5D is a cross-sectional view illustratively showing a state in which an isolation unit is lowered to seal the object to be treated from an external environment according to an exemplary embodiment of the present disclosure.

FIG. 5E illustratively shows an inside of the apparatus for plasma surface treatment according to the exemplary embodiment of the present disclosure.

FIG. 5F is a perspective view illustratively showing an open state of a chamber of the apparatus for plasma surface treatment according to the exemplary embodiment of the present disclosure.

FIG. 6 illustratively shows the isolation unit having at least one surface including a light-transmissive member according to various embodiments of the present disclosure.

FIG. 7 illustratively shows a direction in which plasma discharge occurs as an electric field is formed in an isolation space according to an exemplary embodiment of the present disclosure.

FIG. 8 illustratively shows a state in which a region reacting with plasma in the isolation space dynamically changes according to an exemplary embodiment of the present disclosure.

FIG. 9 illustratively shows a light-transmissive member and a conductive member in the isolation space according to various embodiments of the present disclosure.

FIG. 10 illustratively shows a light-transmissive member and a conductive member in the isolation space according to various embodiments of the present disclosure.

FIG. 11 illustratively shows an operation of a controller of the apparatus for plasma surface treatment according to an exemplary embodiment of the present disclosure.

### DETAILED DESCRIPTION OF EMBODIMENTS OF THE DISCLOSURE

Various embodiments will be now described with reference to the drawings. In the present specification, various descriptions are presented for understanding of the present disclosure. However, it is apparent that these exemplary embodiments can be implemented without the specific descriptions.

Specific structural or functional descriptions corresponding to exemplary embodiments according to the features of the present disclosure are presented for illustrative purposes, and the scope of rights according to such specific structural or functional descriptions is not limited to the examples described in the present disclosure and may encompass various implementable forms, including all equivalents or substitutes falling within the spirit of the present disclosure.

In addition, the term “or” is intended to mean an inclusive “or” rather than an exclusive “or”. That is, unless otherwise specified or clear from the context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in the present specification designates and includes all available combinations of one or more items among enumerated related items.

Additionally, the terms “comprise” and/or “having” should be understood to mean that the corresponding feature and/or element is present. In addition, the terms “comprise” and/or “having” should not be understood as excluding the presence or addition of one or more other features, constitutional elements, and/or groups thereof. Further, unless otherwise specified or in cases where it is not clear from the context to designate a singular form, the singular form in the present specification and claims should be interpreted as meaning “one or more” in general.

The term “at least one of A or B” should be interpreted to mean “a case wherein only A is included”, “a case where only B is included”, or “a case where A and B are combined”.

The description of the presented exemplary embodiments has been provided to allow one skilled in the art to use or implement the present invention. Various modifications to the exemplary embodiments will be apparent to one skilled in the art. The general principles defined herein may be applied to other exemplary embodiments without departing from the scope of the present disclosure. Therefore, the present invention is not limited to the exemplary embodiments presented herein. The present invention should be interpreted within the broadest scope consistent with the principles and novel features presented herein.

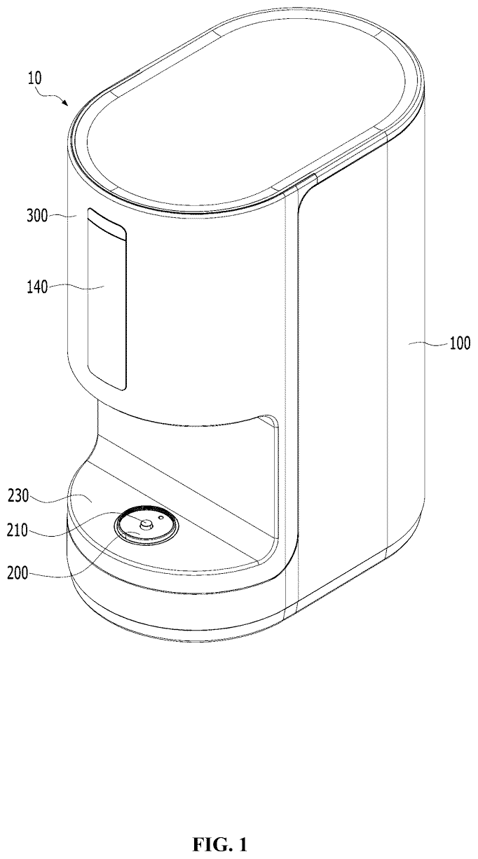

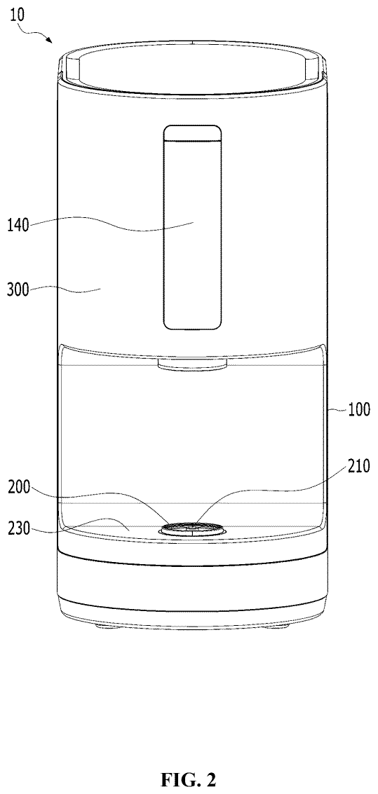

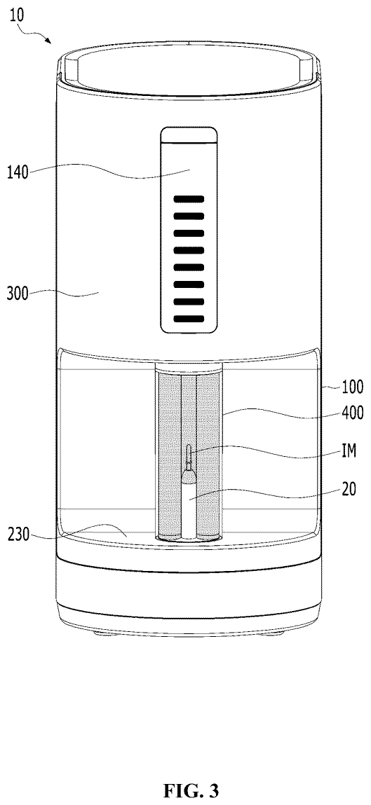

FIG. 1 is a perspective view illustratively showing an apparatus 10 for plasma surface treatment according to an exemplary embodiment of the present disclosure. FIG. 2 is a front view illustratively showing the apparatus for plasma surface treatment according to the exemplary embodiment of the present disclosure. FIG. 3 is a front view illustratively showing a state in which an object to be treated and a gripping device are placed on a placement unit of the apparatus for plasma surface treatment according to the exemplary embodiment of the present disclosure.

In an exemplary embodiment, the apparatus 10 for plasma surface treatment may be used to reduce a level of carbon contamination on a surface of an object to be treated (IM), such as a dental implant, by performing plasma surface treatment under a low-pressure atmospheric state, for example. For example, the apparatus 10 for plasma surface treatment can efficiently remove impurities on the surface of the object to be treated (IM) by performing plasma surface treatment under a vacuum state.

In an exemplary embodiment, the apparatus 10 for plasma surface treatment may include a main body 100 defining an outer shape, an upper member 300 connected to one surface of the main body 100 or integrated with the main body 100 to constitute at least one surface of the main body 100, and a lower member 230 connected to at least one surface of the main body 100 or integrated with the main body 100 to constitute at least one surface of the main body 100. As illustrated in FIG. 1, the upper member 300 represents a member or frame located relatively above in a state in which the main body 100 is placed on the floor, and the lower member 230 represents a member or frame located relatively below in the state in which the main body 100 is placed on the floor. As an example, the upper member 300 and the lower member 230 may be assembled or combined with each other, or may be integrated into one piece to constitute the main body 100.

In an exemplary embodiment, a placement unit 200 may be present on at least one surface of the lower member 230. The placement unit 200 may form a space in which at least one of the object to be treated (IM), a container (not shown) for storing the object to be treated (IM), and/or a gripping device 20 for gripping the object to be treated (IM) is placed.

In an exemplary embodiment, a coupling member 210 may be present in a partial region of the placement unit 200. The coupling member 210 may have a shape capable of coupling with at least one of the object to be treated (IM), the gripping device 20 for gripping the object to be treated (IM), and/or the container (not shown) for storing the object to be treated (IM). A hole may be considered as an example of the coupling member 210. A protrusion may be considered as another example of the coupling member 210. An electrode may be considered as another example of the coupling member 210. A magnet may be considered as another example of the coupling member 210. The coupling member 210 has a shape corresponding to a shape of at least one of the object to be treated (IM), the gripping device (20) for gripping the object to be treated (IM), and/or the container (not shown) for storing the object to be treated (IM), which are a coupling target, making it possible to maintain a fixed state on the placement unit 200 by enhancing coupling strength when at least one of the object to be treated (IM), the gripping device (20) for gripping the object to be treated (IM), and/or the container (not shown) for storing the object to be treated (IM) is placed on the placement unit 200.

In an exemplary embodiment, a display unit 140 may be formed on one surface of the upper member 300. For example, information about an operation and/or state of the apparatus 10 for plasma surface treatment may be output through the display unit 140. As another example, information about a state, a treatment process, and/or a treatment result of the object to be treated (IM) according to the operation of the apparatus 10 for plasma surface treatment may be output through the display unit 140. The display unit 140 may provide information about the apparatus 10 and/or the object to be treated (IM) to the user by outputting an image and/or illumination. By way of example, and not limitation, the display unit 140 may include a liquid crystal display (LCD), a thin film transistor-liquid crystal display (TFT LCD), an organic light-emitting diode (OLED), a flexible display and/or a 3D display. Some of the above-described example configurations of the display unit 140 may be transparent or light-transmissive so that the outside can be viewed through the same. This may be referred to as a transparent display, and representative examples of the transparent display include TOLED (Transparent OLED).

As illustrated in FIG. 3, in an exemplary embodiment, the display unit 140 may output a plurality of bar-shaped indicators to externally display a progress of plasma surface treatment for the object to be treated (IM).

As illustrated in FIGS. 1, 2, and 3, the apparatus 10 for plasma surface treatment according to an exemplary embodiment of the present disclosure may include a placement unit 200 on which an object to be treated (IM) is placed, an isolation unit 400 configured to move relative to the placement unit 200 to form an isolation space in which an inside is sealed from an external environment, an upper member 300 disposed above the placement unit 200 and capable of accommodating at least a part of the isolation unit 400, and a lower member 230 located relatively below with respect to the upper member 300 and forming the placement unit 200 in at least a partial region (for example, upper surface).

In an exemplary embodiment, the placement unit 200 may be located on one surface (for example, a front surface) of the main body 100. The placement unit 200 may be located in a direction opposite to the upper member 300. For example, the placement unit 200 may be located below the upper member 300. The placement unit 200 may be located on the lower member 230. By way of example, and not limitation, the lower member 230 and the upper member 300 may have a shape that protrudes forward, and a space in which the object to be treated (IM) can be placed may be formed between the upper member 300 and the lower member 230. An isolation space may be formed resulting from relative movement between the isolation unit 400 and the placement unit 200 in a space of the main body 100 where the object to be treated (IM) can be placed. For example, the isolation space may mean a sealed space in which an inside is sealed from an external environment.

As an example, the isolation space may mean a vacuum chamber that is formed as the placement unit 200 and the isolation unit 400 come into contact with each other via an elastic member (e.g., a silicone cover, or the like) provided in at least one of the placement unit 200 and the isolation unit 400. The inside of the vacuum chamber is in a low-pressure state of 10 torr or lower, and when a high voltage of up to 3 kV is applied, a surface of the object to be treated (IM) can be excited. A high voltage may be applied into the vacuum chamber through a power supply unit connected to an upper part of the isolation unit 400, and plasma surface treatment may be performed in the vacuum chamber in a manner of exciting the object to be treated (IM) through an electrode or a ground part connected to a lower part of the isolation unit 400 or an upper part of the placement unit 200. The exciting operation on the object to be treated (IM) can efficiently remove impurities on the surface of the object to be treated (IM). In addition, the impurities on the surface of the object to be treated (IM) can be efficiently removed using a pressure difference generated inside the vacuum chamber by a vacuum pump.

In an exemplary embodiment, the upper surface of the placement unit 200 may be provided with a coupling member 210 into which a lower part of at least one of the object to be treated (IM), the gripping device 20 for gripping the object to be treated (IM), and/or the container (not shown) for storing the object to be treated (IM) is inserted. At least one of the object to be treated (IM), the gripping device 20 for gripping the object to be treated (IM), and/or the container (not shown) for storing the object to be treated (IM) connected to the placement unit 200 via the coupling member 210 may be fixed to the placement unit 220. Additionally, the placement unit 200 may be provided with a magnet. The magnet may be formed on a bottom surface of the coupling member 210. At least one of the object to be treated (IM), the gripping device 20 for gripping the object to be treated (IM), and/or the container (not shown) for storing the object to be treated (IM) may be coupled with the coupling member 210 more easily and more firmly by the magnet 220. In this regard, the object to be treated (IM), the gripping device 20 for gripping the object to be treated (IM), and/or the container (not shown) for storing the object to be treated (IM) that can be coupled with the coupling member 210 may include a metal material in at least a part thereof, making it possible to facilitate the coupling with the magnet. The apparatus 10 according to an exemplary embodiment of the present disclosure is provided with a magnet, making it possible to further facilitate the coupling between the placement unit 200 and at least one of the object to be treated (IM), the gripping device 20 for gripping the object to be treated (IM), and/or the container (not shown) for storing the object to be treated (IM). Such easiness of the coupling makes it possible to increase the convenience of a user who performs plasma surface treatment and the user experience.

In an exemplary embodiment, the coupling member 210 present on the upper part of the placement unit 200 may have various shapes depending on aspects of implementation. Examples of such a coupling member may include a coupling member using a hole, a coupling member using a magnet, a coupling member using a protrusion, a coupling member using an adhesive material, a coupling member using a belt, and/or a coupling member using tongs.

In an exemplary embodiment, the isolation unit 400 may move relative to the placement unit 200 to isolate or seal the object to be treated (IM) from the external environment. The relative movement of the isolation unit 400 and the placement unit 200 may include at least one of, for example, a first movement method in which the isolation unit 400 moves in a direction of the placement unit 200, a second movement method in which the placement unit 200 moves in a direction of the isolation unit 400, and a third movement method in which the isolation unit 400 and the placement unit 200 move toward each other. The isolation unit 400 can form by combining with the placement unit 200 an isolation space in which an inside is sealed against the external environment by moving relative to the placement unit 200. The isolation unit 400 and the placement unit 200 may move relative to each other and come into contact with each other to form an isolation space with a sealed inside. For example, as at least one of the isolation unit 400 and the placement unit 200 is raised and lowered, one surface of the isolation unit 400 comes into contact with one surface of the placement unit 200, so that an inside of the isolation unit 400 may be formed with an isolation space. As an example, the isolation unit 400 may refer to a tube-shaped or rectangular parallelepiped member that can move toward the placement unit 200 in response to external force.

In an exemplary embodiment, the upper member 300 may accommodate at least a part (e.g., all) of the isolation unit 400. The isolation unit 400 can move up and down between a sealed position in which the isolation unit is in contact with the placement unit 200 and an accommodation position in which the isolation unit is accommodated inside the upper member 300.

In an exemplary embodiment, the isolation unit 400 may include one or more outer walls. In an exemplary embodiment, the isolation unit 400 may be configured as a dual structure including an inner wall and an outer wall. In an exemplary embodiment, both the inner wall and the outer wall of the isolation unit 400 may be made of a transparent material. In an exemplary embodiment, both the inner wall and the outer wall of the isolation unit 400 may include a light-transmissive member, and at least one of the inner wall and the outer wall may include a conductive member. In an exemplary embodiment, a hollow portion may be formed at one location (for example, a center) of the isolation unit 400. When the isolation unit 400 moves and, accordingly, the lower surface of the isolation unit 400 comes into contact with the upper surface of the placement unit 200, the hollow portion of the isolation unit 400 may form an isolation space. The isolation space in the present disclosure may mean a space formed resulting from the relative movement of the isolation unit 400 and the placement unit 200. For example, the isolation space can protect the object to be treated (IM) present inside the isolation space from the external environment. For example, the isolation space may seal the object to be treated (IM) present inside the isolation space from the external environment. When the isolation unit 400 and the placement unit 200 come into contact with each other to form an isolation space, at least one of the object to be treated (IM), the gripping device 20 for gripping the object to be treated (IM), and/or the container (not shown) for storing the object to be treated (IM) is located in the isolation space.

In an exemplary embodiment, an electrode may be located in at least one region of the isolation unit 400. For example, the electrode may be located between an inner surface and an outer surface of the isolation unit 400. As an example, the electrode may be formed in a manner of being coated on an outer surface of an inner wall of the isolation unit and an inner surface of an outer wall. The electrode present in the isolation unit 400 may include a transparent electrode. Such an electrode may be configured to provide electrical conductivity to the isolation unit 400. In an exemplary embodiment, when the isolation unit 400 and the placement unit 200 form an isolation space, the electrode may be formed or disposed to correspond to a shape of the object to be treated (IM). As an example, when an isolation space is formed, the electrode may be configured to surround the object to be treated (IM).

In an exemplary embodiment, the electrode may be provided in one region of the coupling member 210 of the placement unit 200. When the gripping device 20 is placed while being coupled (for example, inserted) to the coupling member 210 of the placement unit 200, a lower surface of the gripping device 20 may be connected to the electrode. Portions where the gripping device 20 and the object to be treated (IM) are connected may be made of a metal material so that the gripping device 20 and the object to be treated (IM) are electrically connected to each other. Therefore, when the gripping device 20 is placed on the placement unit 200, the electrode connected to the placement unit 200, and the gripping device 20 and the object to be treated (IM) may be electrically connected.

In an exemplary embodiment, at least a part of the electrode connected to the placement unit 200 may be exposed to the inside of the isolation space. The electrode is electrically connected to the object to be treated (IM) or at least a part of the container in which the object to be treated (IM) is stored, which are present in the isolation space, so that the efficiency of the plasma reaction to the object to be treated (IM) can be increased.

In an exemplary embodiment, a coating layer (not shown) may be formed on the isolation unit 400 (for example, an inner surface of the inner wall of the isolation unit). The coating layer may be made of a material that can withstand high temperatures and high voltages in order to prevent the inner surface of the isolation unit 400 from being damaged or foreign matters from being eluted due to high temperatures and high voltages during plasma discharge. For example, the coating layer may be made of a heat-resistant material and/or an insulating material.

In an exemplary embodiment, the coating layer may include a material containing calcium. In this way, when the coating layer is made of a material containing calcium, calcium can be allowed to adhere to the surface of the object to be treated (IM) by inducing elution of calcium by plasma discharge. In this way, by artificially causing calcium to adhere to the surface of the object to be treated (IM), when implanting or engrafting the object to be treated (IM) in the human body, the inflammatory response can be suppressed or alleviated, and furthermore, the implantation or engraftment is allowed to be more robust, ensuring a high implantation rate and/or a high engraftment rate.

In an exemplary embodiment, the coating layer may include a biocompatible material. Since the biocompatible material may adhere to the surface of the object to be treated (IM) during a plasma treatment process, the object to be treated (IM) can be more stably implanted or engrafted in the human body.

In an exemplary embodiment, an inner side of the isolation unit 400 may be made of a chemical-resistant material or formed with a chemical-resistant coating layer. At least a part of the isolation unit 400 is made of a transparent material to allow the atmosphere under a lower pressure state discharged inside the isolation unit 400 to be confirmed with a naked eye from the outside. The transparent material may include, for example, a glass material. By way of example, and not limitation, the inner wall and the outer wall of the isolation unit 400 may have a shape of a tubular tube made of transparent tempered glass.

In an exemplary embodiment, the isolation unit 400 may include a material having elasticity that is deformed so that the inside of the isolation unit 400 is sealed against the external environment using a pressure difference between the inside and the outside of the isolation unit 400. The remaining region of the placement unit 200 excluding the coupling member 210 may include a material having elasticity. The elastic members present in the isolation unit 400 and the placement unit 200 can alleviate impact that is transmitted to the isolation unit 400 and/or the placement unit 200 when the isolation unit 400 is lowered and, accordingly, the lower surface of the isolation unit 400 comes into contact with the upper surface of the placement unit 200. The elastic member can make the seal between the isolation unit 400 and the placement unit 200 more robust. The elastic member can further improve the sealing force between the lower surface of the isolation unit 400 and the upper surface of the placement unit 200 by generating elastic force in the process of contact between the isolation unit 400 and the placement unit 200. In an exemplary embodiment, the remaining region of the region corresponding to the placement unit 200, excluding the coupling member 210 that allows electrical connection, may be composed of an elastic member.

In the present disclosure, for the sake of description, the exemplary embodiment in which the object to be treated (IM) is placed on the placement unit 200 while being gripped by the gripping device 20 has been given as an example. However, depending on the implementation aspect, a case where the object to be treated (IM) itself is placed on the placement unit 200, a case where a container is placed on the placement unit 200 with the object to be treated (IM) stored in the container for storing the object to be treated (IM), or a case where the gripping device 20 for gripping the object to be treated (IM) is placed on the placement unit 200 while gripping the object to be treated (IM) may also be included within the scope of the present disclosure. Therefore, in the present disclosure, the expression that the object to be treated (IM) is placed may be used to encompass that the object to be treated (IM) itself is placed, a container is placed with the object to be treated (IM) stored in the container for storing the object to be treated (IM), or the gripping device 20 for gripping the object to be treated (IM) is placed while gripping the object to be treated (IM).

In an exemplary embodiment, the apparatus 10 may include a placement unit 200 on which the object to be treated (IM) can be placed. The object to be treated (IM) may refer to an object that is subjected to plasma surface treatment. The object to be treated (IM) may include an object of any shape whose surface can be modified through plasma treatment.

By way of example, and not limitation, the object to be treated (IM) may include medical products such as an implant fixture, a bone graft material, an artificial joint, and/or a ratchet. The surface of the object to be treated (IM) may be modified from hydrophobicity to hydrophilicity by plasma surface treatment of the apparatus 10.

In an exemplary embodiment, the object to be treated (IM) may be made of a conductive material. When plasma surface treatment is performed for the object to be treated (IM), the object to be treated (IM) may be operated as an electrode capable of forming an electric field for generating plasma. Therefore, because the object to be treated (IM) can operate as an electrode, a dielectric layer clogging effect can be reduced. For example, the object to be treated (IM) may be made of a material such as titanium that is highly harmless to the human body and is easy to synostosis with osseous tissue.

In an exemplary embodiment, the object to be treated (IM) may be composed of a structure that can be inserted into the alveolar bone and support an artificial tooth. For example, the object to be treated (IM) may have a shape extending in an up-down direction. For example, the object to be treated (IM) may have a pillar shape.

In an exemplary embodiment, the object to be treated (IM) may be configured to have an external shape such as a groove, a screw, or a protrusion such that a contact area is increased when coupled with an object to be coupled. For example, the object to be treated (IM) may have a shape having an outer diameter expanding or decreasing in one direction.

In an exemplary embodiment, the placement unit 200 may have any structure that can accommodate the object to be treated (IM). For example, the placement unit 200 may have a flat shape at a portion that contacts the object to be treated (IM) to accommodate the object to be treated (IM). The placement unit 200 has a shape in which the portion to be in contact with the object to be treated (IM) is substantially horizontal to the ground, making it possible to maintain more stably a state in which the object to be treated (IM) is placed. As another example, the placement unit 200 may have a shape in which a portion (e.g., the upper surface of the placement unit 200) that contacts the object to be treated (IM) to accommodate the object to be treated (IM) corresponds to a shape of a portion (for example, one end portion of the object to be treated (IM)) of the object to be treated (IM) to be in contact with the placement unit 200. In this example, when one end portion of the object to be treated (IM) has a concave shape (or convex shape), the portion of the placement unit 200 to be in contact with the object to be treated (IM) may have a convex shape (or concave shape) corresponding to the concave shape (or convex shape). The placement unit 200 may be made of a plastic material and/or a metal material. For example, at least a region of the placement unit 200, which forms the isolation space, may be made of a heat-resistant or insulating material.

In an exemplary embodiment, the placement unit 200 may have a coupling member or a storage hole corresponding to a shape of the object to be treated (IM), the gripping device 20 for gripping the object to be treated (IM), and/or the container in which the object to be treated (IM) is stored. An electrode may be disposed on an inner peripheral surface or outer peripheral surface of the coupling member 210, and when the isolation unit 400 and the placement unit 200 form an isolation space, at least a part of the electrode may be present exposed inside the isolation space. The electrode exposed inside the isolation space may be electrically connected to the object to be treated (IM), the gripping device 20 for gripping the object to be treated (IM), and/or a part of the container in which the object to be treated (IM) is stored. The electrode of the placement unit 200 may have magnetism. Since a part of the electrode has magnetism, coupling may be made using the magnetism between the electrode and the object to be treated (IM), the gripping device 20 for gripping the object to be treated (IM), and/or the container in which the object to be treated (IM) is stored.

In an exemplary embodiment, the object to be treated (IM) itself may be accommodated or placed on the placement unit 200. As another example, the object to be treated (IM) may be accommodated or placed on the placement unit 200 while being gripped by the gripping device 20. As another example, the object to be treated (IM) may be accommodated or placed on the placement unit 200 while being stored in a separate container. The container for storing the object to be treated (IM) or the gripping device 20 for gripping the object to be treated (IM) may include a fastening means for fastening the object to be treated (IM) so that the object to be treated (IM) can be maintained placed on the placement unit 200. By way of example, and not limitation, the fastening means may include a fastening means using magnetism, a fastening means using a pin and an opening portion, a screw-type fastening means using a screw, a flange-type fastening means using a connector and a connecting clip, and a screw tightening-type fastening means, and/or a fastening means using an adhesive.

In an exemplary embodiment, the object to be treated (IM) may be taken out by the gripping device 20 from a packaging in which the object to be treated (IM) is sealed. Accordingly, the gripping device 20 can be placed on the placement unit 200 while gripping the object to be treated (IM). That is, the object to be treated (IM) may be placed on the placement unit 200 by the gripping device 20. In this case, one end of the gripping device 20 may have a shape for gripping the object to be treated (IM), and the other end of the gripping device 20 may have a flat shape for being placed on the placement unit 200 or a shape corresponding to an end portion of the placement unit 200.

In an exemplary embodiment, the gripping device 20 may be connected to the placement unit 200 of the apparatus 10 for plasma surface treatment while accommodating the object to be treated (IM) on one surface. Accordingly, the gripping device 20 can transmit power from the apparatus 10 to the object to be treated (IM) while stably fixing the object to be treated (IM) to the apparatus 10, so that surface treatment can be effectively performed for the object to be treated (IM).

In an exemplary embodiment, the gripping device 20 may include a housing member that defines an outer shape of the gripping device 20, and one or more magnet members. The housing member may have an accommodation part having a shape capable of accommodating objects to be treated (IM) with different diameters or sizes. The inside of the accommodation part has a concave shape, making it possible to facilitate the accommodation of the object to be treated (IM). The accommodation part of the housing member may have a first diameter at a position corresponding to a first height of the housing member and a second diameter at a position corresponding to a second height of the housing member. Accordingly, various types of objects to be treated (IM) with different sizes can be easily coupled with the gripping device 20. The housing member may be made of a conductive material, so that the power supply unit and/or one or more electrodes of the apparatus 10 and the object to be treated (IM) may be electrically connected.

In an exemplary embodiment, the housing member of the gripping device 20 may include a magnet member, so that the object to be treated (IM) accommodated in the accommodation part may be connected to the magnet member. The object to be treated (IM) accommodated in the accommodation part can be firmly fixed in the housing member through the magnet member. Additionally, the magnet member may be utilized to solidify the connection between the gripping device 20 and the apparatus 10. The magnet member in the present disclosure may include a magnetic member or a member made of a material (for example, a member including iron, a member including chromium plating, or the like) that is affected by magnetism of another magnetic member.

In an exemplary embodiment, a side or one end portion of the housing member of the gripping device 20 connected to the apparatus 10 may have a shape that can be connected to an electrode of the apparatus 10 (for example, an electrode connected to the placement unit 200). As an example, a part of the gripping device 20 connected to the apparatus 10 may have a shape corresponding to a shape of the placement unit 200 or a shape of the electrode.

According to an exemplary embodiment of the present disclosure, a container for storing the object to be treated (IM) may be connected to the placement unit 200 of the apparatus 10. The container may be configured to surround the object to be treated (IM). The container can seal the stored object to be treated (IM) from the outside.

In an exemplary embodiment, the container may have, for example, one or more holes (for example, two holes), so that a movement path of plasma may be formed through the one or more holes when performing surface treatment for the object to be treated (IM). Air inside the container may be exhausted through the one or more holes formed in the container. The inside of the container may be vacuumed through one or more holes formed in the container prior to plasma surface treatment. By way of example, and not limitation, a first hole may be formed on one surface of an inner cover of the container and a second hole may be formed on one surface of an outer cover of the container. As an example, these holes may be formed in a direction in which they do not overlap each other. These holes may be formed along a height direction of the container. As another example, these holes may be formed in a shape in which they do not overlap each other along any axis of X, Y, and Z axes of the container.

In an exemplary embodiment, the container may have an inner cover and an outer cover. The inner cover can store the object to be treated (IM) therein, and the outer cover can surround the inner cover. The object to be treated (IM) can be more easily protected inside the container through at least two covers. At least one of the inner cover and the outer cover may be made of an insulating material, including, for example, a resin material. The inner cover and the outer cover may be made of a light-transmissive material so that the object to be treated (IM) stored therein can be confirmed from the outside.

As described above, the container, like the gripping device 20, may have a shape capable of coupling with the placement unit 200.

In an exemplary embodiment of the present disclosure, the object to be treated (IM) may include a non-conductor such as a bone graft material or food seed. In an exemplary embodiment, the container for storing the object to be treated (IM) may include an electrode member disposed near a storage space in which the object to be treated (IM) is stored. In addition, the container for storing the object to be treated (IM) may further include a region having a different dielectric constant and connected to the electrode member. In an exemplary embodiment, the container includes a concave groove-shaped storage space inside the outer surface of the container, thereby allowing plasma discharge to occur around the object to be treated (IM). In an exemplary embodiment, the container can efficiently perform plasma discharge for the object to be treated (IM) by reducing the electrical resistance inside the container through a groove surrounding the inside of the outer surface of the container. Since plasma may have difficulty in discharging around a non-conductor with high electrical resistance, when the object to be treated (IM) is a non-conductor, it is necessary to lower the electrical resistance around the non-conductor. The container according to an exemplary embodiment may include region(s) having different dielectric constants to allow plasma to be easily generated around the object to be treated (IM). That is, the container can be divided into a region with a low dielectric constant and a region with a high dielectric constant, so that plasma discharge can be concentrated on the object to be treated (IM) inside the space within the container. In addition, regions with different dielectric constants may be connected to an electrode member in the container to induce plasma discharge into the storage space within the container. By way of example, and not limitation, the container may be made of a transparent dielectric material, allowing a discharge process of the plasma to be confirmed from the outside.

In an exemplary embodiment, at least a part of the storage container may include a light-transmissive member so that when the object to be treated (IM) is placed on the placement unit 200 while stored in the storage container, the object to be treated (IM) is visible from the external environment during the operation period of the apparatus 10. Accordingly, the process of plasma treatment for the object to be treated (IM) stored in the storage container can be confirmed from the outside. In additional exemplary embodiments, at least a part of the storage container may include a conductive material. Accordingly, the efficiency of plasma surface treatment for the object to be treated (IM) stored in the storage container can be maximized.

In an exemplary embodiment, at least a part of the gripping device 20 may include a light-transmissive member so that when the object to be treated (IM) is placed on the placement unit 200 while gripped by the gripping device 20, the object to be treated (IM) is visible from the external environment during the operation period of the apparatus 10. Here, at least a part of the gripping device 20 may refer to a partial region of the gripping device 20, which shields the object to be treated (IM) when viewed from the outside, among regions of the gripping device 20. For example, at least a part of the gripping device 20 may refer to a surface at a position (e.g., height) corresponding to a position (e.g., height) on the gripping device 20 at which the object to be treated (IM) is present. Accordingly, even in a situation where the outer shape of the gripping device 20 covers the object to be treated (IM), the process of plasma surface treatment for the object to be treated (IM) can be confirmed with a naked eye from the outside due to the transparency of the gripping device 20.

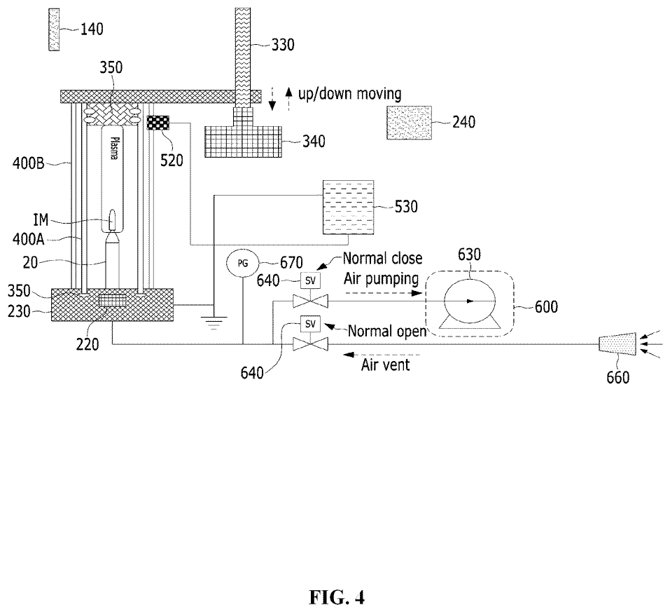

FIG. 4 is a conceptual diagram illustratively showing the apparatus 10 for plasma surface treatment according to the exemplary embodiment of the present disclosure.

To prevent redundant description, the description of the components described above in FIGS. 1 to 3 among the components of the apparatus 10 for plasma surface treatment illustrated in FIG. 4 will be omitted.

In an exemplary embodiment, the apparatus 10 for plasma surface treatment may include a power supply unit 530. The power supply unit 530 may supply power to the components of the plasma surface treatment apparatus 10 so that the components of the plasma surface treatment apparatus 10 can operate. As an example of the power supply unit 530, a high voltage power supply (HVPS) may be considered.

In an exemplary embodiment, the power supply unit 530 may generate power to be applied to a plurality of electrodes within the plasma surface treatment apparatus 10. As an example, the power supply unit 530 may form an electric field during a process time for which an internal pressure of the isolation unit 400 is within a preset process pressure range, thereby generating alternating current power (AC) for discharging the atmosphere under a lower pressure state. Depending on the implementation aspect, a plasma reaction in the present disclosure may encompass a plasma reaction generated by applying a direct current voltage, a plasma reaction generated by applying RF or microwave, and/or an inductively coupled plasma (ICP) reaction, and the like.

In an exemplary embodiment, the apparatus 10 may include a controller 240. The controller 240 may control operations of the components of the apparatus 10 based on the power supplied by the power supply unit 530. Accordingly, each component of the apparatus 10 may be operated under control of the controller 240 based on the power supplied by the power supply unit 530.

In an exemplary embodiment, the power supply unit 530 may supply power necessary for an exhaust unit 600 to perform an exhaust operation. In an exemplary embodiment, the controller 240 may control the exhaust operation of the exhaust unit 600. In an exemplary embodiment, the exhaust unit 600 may exhaust the inside of the isolation unit 400. The exhaust unit 600 may exhaust the atmosphere inside the isolation unit 400. The exhaust unit 600 may exhaust the atmosphere inside the isolation unit 400 to make the isolation space into a low-pressure atmospheric state or a vacuum state. The exhaust unit 600 may adjust the internal pressure of the isolation unit 400 to be within a preset process pressure range. By way of example, and not limitation, the exhaust unit 600 may include any type of member capable of communicating the atmosphere, and as an example, the exhaust unit 600 may include a mechanical or electric air intake for sucking air. By way of example, and not limitation, the preset process pressure range may be a range of 0.001 Torr or higher and lower than 100 Torr. As another example, the preset process pressure range may be a range of 1 Torr or higher and lower than 30 Torr. The exhaust unit 600 may include an exhaust pump 630 that generates suction force, a pump valve 640 that interrupts communication between the exhaust pump 630 and an exhaust flow passage, a filter 660 that filters out foreign matters on the exhaust flow passage, and/or a pressure sensor 670 that measures a pressure on the exhaust flow passage. The pressure sensor 670 may measure a pressure of the isolation unit 400 and/or the exhaust flow passage 620. By way of example, and not limitation, the pressure sensor 670 may include any type of device that outputs a magnitude of fluid pressure applied to the pressure sensor 670 as an electrical signal. The pressure sensor 670 may output a pressure in pascal units needed to prevent a fluid from expanding and measure an absolute pressure, a gauge pressure, and/or a differential pressure.

In an exemplary embodiment, the exhaust pump 630 may be used to transfer gas from a specific space to another space. The exhaust pump 630 may be provided on the exhaust flow passage to exhaust the air inside the isolation unit 400 to the outside of the isolation space upon actuation. For example, the exhaust pump 630 may continuously perform an exhaust operation to maintain the internal pressure of the isolation unit 400 constant (e.g., in a low-pressure state), and plasma discharge may be performed in the isolation space of the isolation unit 400 maintained in the low-pressure state. As another example, the exhaust pump 630 may perform an exhaust operation for varying the internal pressure of the isolation unit 400, and accordingly, the plasma discharge may be performed in the isolation space of the isolation unit 400 while the atmospheric pressure is varied.

In an exemplary embodiment, the pump valve 640 may be provided on the exhaust flow passage to perform an opening operation (for example, normal open) and a closing operation (for example, normal close). When the pump valve 640 is opened, the exhaust pump 630 and the exhaust flow passage may be in communication with each other. When the pump valve 640 is closed, communication between the exhaust pump 630 and the exhaust flow passage may be interrupted. Therefore, when the exhaust pump 630 is actuated with the pump valve 640 open, the air inside the isolation unit 400 can be exhausted, and when the pump valve 540 is closed, the air inside the isolation unit 400 is not exhausted even if the exhaust pump 630 is actuated. By way of example, and not limitation, the pump valve 640 may include a solenoid valve whose opening and closing operations can be performed by a controller (not shown). For example, the internal pressure of the isolation unit 400 may be maintained constant according to the opening and closing operations of the pump valve 640, and plasma discharge may be performed inside the isolation unit 400 in which the pressure of the atmosphere under a lower pressure state is maintained constant. As another example, the internal pressure of the isolation unit 400 may vary according to the opening and closing operations of the pump valve 640, and accordingly, plasma discharge may be performed in the isolation space of the isolation unit 400 while the atmospheric pressure is varied.

In an exemplary embodiment, a HEPA filter may be considered as an example of the filter 660.

In an exemplary embodiment of the present disclosure, the power supply unit 530 may supply power necessary for a motor 340 to perform operations for relative movement of the isolation unit 400 and/or the placement unit 200. In an exemplary embodiment, the controller 240 may control the operation of the motor 340. As an example, the motor 340 may include a step motor. More specifically, motion of the motor 340 may cause a shaft 330 to rotate. That is, the motion of the motor 340 can be converted into rotational movement of the shaft 330. The rotational movement of the shaft 330 may cause the isolation unit 400 to move up and down. As the isolation unit 400 moves down, the lower part of the isolation unit 400 may be coupled with the upper surface of the placement unit 200. In addition, as the isolation unit 400 moves up, the coupling between the isolation unit 400 and the placement unit 200 may be released. In this coupling process, one or more elastic members 350 present in the placement unit 200 and one or more elastic members 350 present in the isolation unit 400 can alleviate impact in the process of movement, coupling and decoupling of the isolation unit 400 and maintain the coupling between the isolation unit 400 and the placement unit 200 more firmly.

In an exemplary embodiment, the isolation unit 400 may be composed of, for example, a first tube 400A of Pyrex glass and a second tube 400B for a protective function. The first tube 400A may be present inside the second tube 400B. The first tube 400A and the second tube 400B may have different diameters but have shapes corresponding to each other. Both the first tube 400A and the second tube 400B may include transparent members. As illustrated in FIG. 4, a space formed by the first tube 400A may correspond to the isolation space. Upper and lower portions of the first tube 400A are connected to the elastic members 350, so that the impact during the movement of the isolation unit 400 can be alleviated, and a degree of sealing of the isolation space formed by the first tube 400A can be further enhanced. According to an exemplary embodiment of the present disclosure, an electrode 520 may be located on an inner surface of the first tube 400A or between the first tube 400A and the second tube 400B. Such an electrode 520 may be formed in a coating manner. The electrode 520 may include a transparent electrode. Accordingly, the efficiency of the plasma surface treatment for the object to be treated (IM) can be increased while ensuring visibility of the plasma surface treatment process for the object to be treated (IM) inside the isolation space.

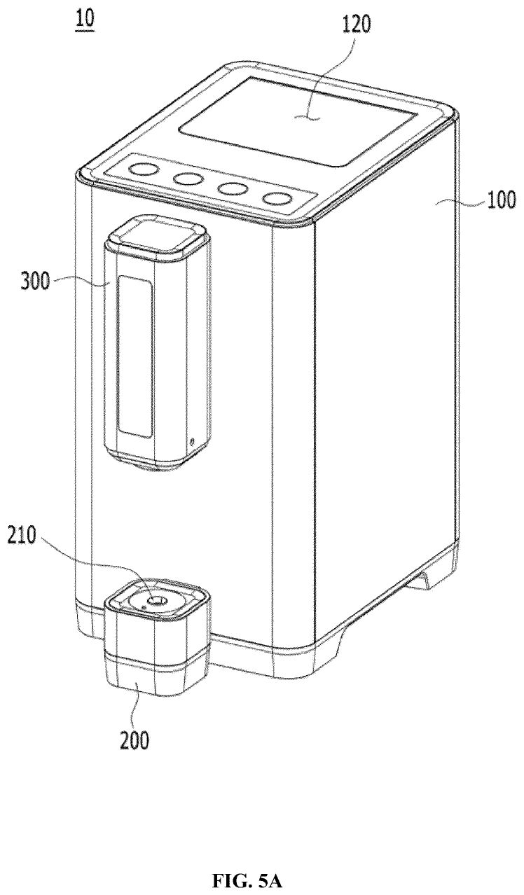

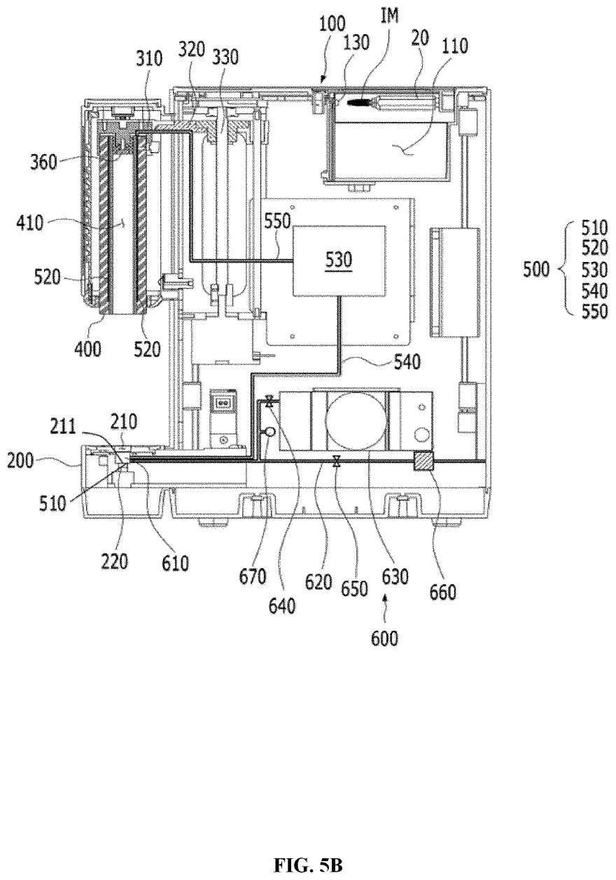

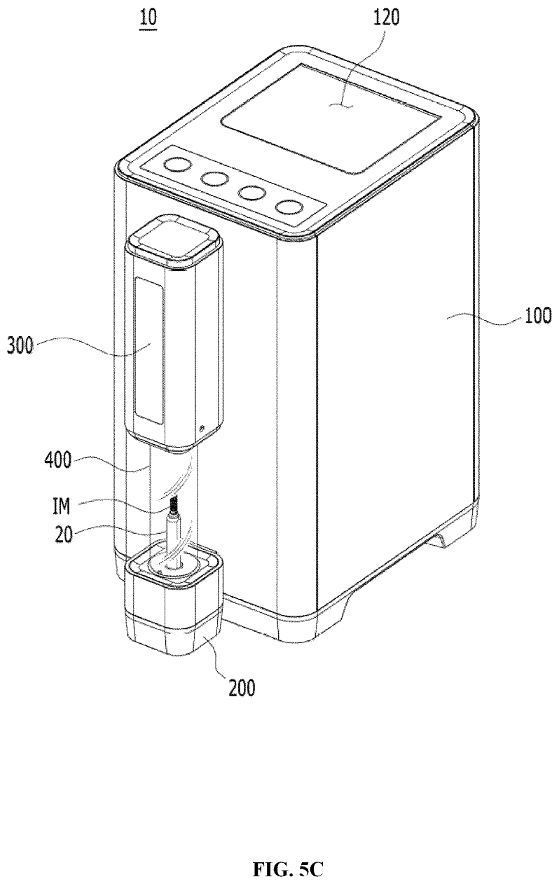

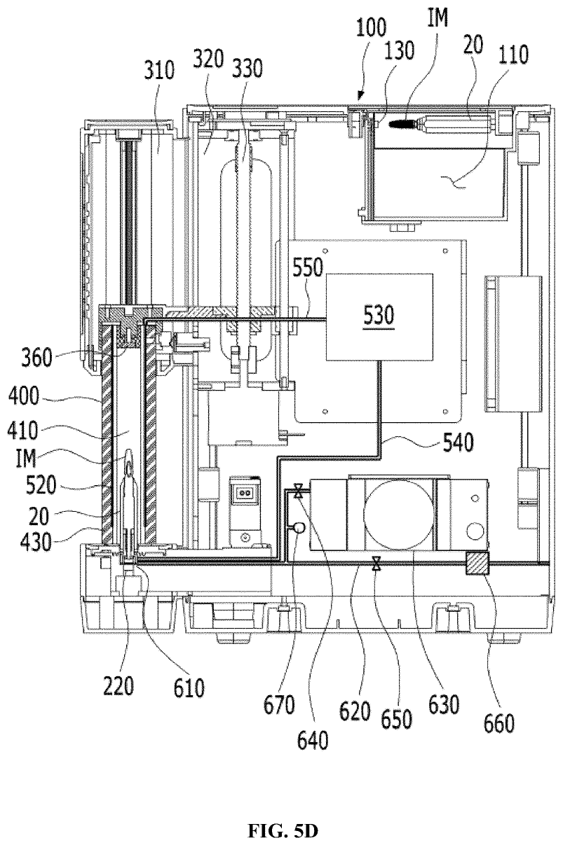

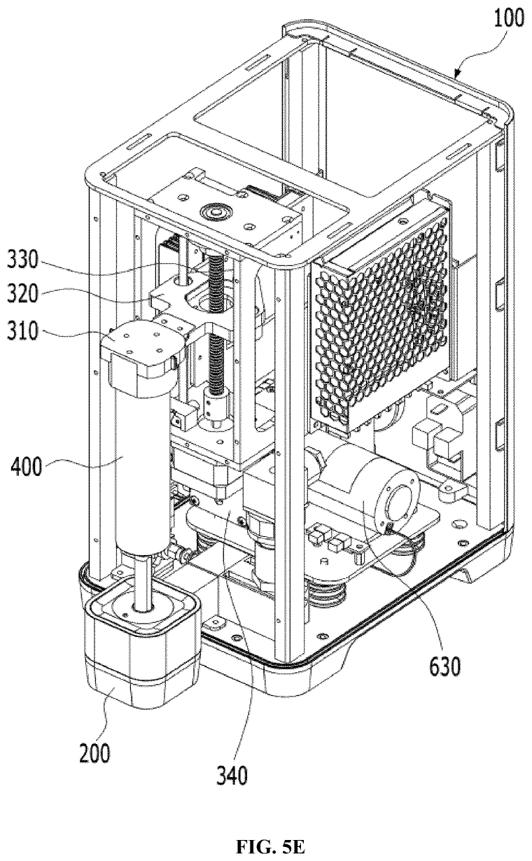

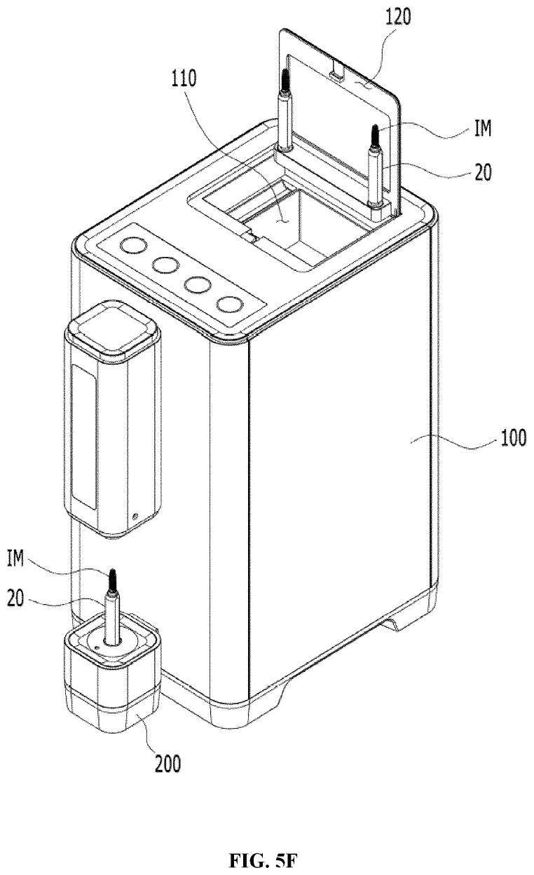

FIG. 5A is a perspective view illustratively showing the apparatus 10 for plasma surface treatment according to the exemplary embodiment of the present disclosure. FIG. 5B is a cross-sectional view illustratively showing the apparatus 10 for plasma surface treatment according to the exemplary embodiment of the present disclosure.

The apparatus 10 for plasma surface treatment shown in exemplary embodiment of FIGS. 5A to 5F may correspond to the exemplary embodiment shown in FIGS. 1 to 4 from a standpoint of ensuring visibility of plasma surface treatment while increasing the efficiency of the plasma surface treatment. The illustrations of FIGS. 5A to 5F are used for illustrative purposes, and the apparatus 10 for plasma surface treatment having various outer shapes and structures can be conceived according to implementation aspects while maintaining the core idea of the present disclosure.

In an exemplary embodiment, the apparatus 10 for plasma surface treatment may include a main body 100 that forms an outer shape of the apparatus 10, an upper member 300 connected to one surface of the main body 100, a chamber door 120 that can cause a chamber 110 of the main body 100 to be opened and closed, and a coupling member 210 that can be coupled with at least one of an object to be treated (IM), a gripping device 20 for gripping the object to be treated (IM), and/or a container (not shown) for storing the object to be treated (IM). The coupling member 210 may have a shape capable of coupling with at least one of the object to be treated (IM), the gripping device 20 for gripping the object to be treated (IM), and/or the container (not shown) for storing the object to be treated (IM). A hole may be considered as an example of the coupling member 210. A protrusion may be considered as another example of the coupling member 210. An electrode may be considered as another example of the coupling member 210. A magnet may be considered as another example of the coupling member 210. The coupling member 210 has a shape corresponding to a shape of at least one of the object to be treated (IM), the gripping device (20) for gripping the object to be treated (IM), and/or the container (not shown) for storing the object to be treated (IM), which are a coupling target, making it possible to maintain a fixed state on the placement unit 200 by enhancing coupling strength when at least one of the object to be treated (IM), the gripping device (20) for gripping the object to be treated (IM), and/or the container (not shown) for storing the object to be treated (IM) is placed on the placement unit 200.

The apparatus 10 for plasma surface treatment according to an exemplary embodiment of the present disclosure may include a placement unit 200 on which an object to be treated (IM) is placed, an isolation unit 400 configured to move relative to the placement unit 200 to form an isolation space in which an inside is sealed from an external environment, a treatment unit 500 configured to allow plasma discharge to occur by forming an electric field inside the isolation space, an exhaust unit 600 configured to exhaust an atmosphere inside the isolation unit 400 to form an atmosphere under a lower pressure state within a preset process pressure range inside the isolation unit 400, an upper member 300 disposed above the placement unit 200 and capable of accommodating at least a part of the isolation unit 400, an elevating part 310 provided inside the upper member 300 and connected to the isolation unit 400, a motor 340 configured to raise and lower the elevating part 310 in order to raise and lower the isolation unit 400, an elastic member 350 provided between the elevating part 310 and the isolation unit 400, a magnet 220 provided in the placement unit 200, a chamber 110 provided at an upper part of a main body 100, a first UV light source unit 130 configured to irradiate an inside of the chamber 130 with ultraviolet (UV), a chamber door 120 for opening and closing the inside of the chamber 110, and/or a second UV light source unit 360 configured to irradiate the inside of the isolation space formed by the isolation unit 400 with ultraviolet (UV). Depending on the implementation aspect, at least some of the components of the apparatus 10 may be omitted or at least some of components may be added to the apparatus 10.

In the present disclosure, the isolation space may be used to mean a space that can be at least partially isolated, blocked, or protected from the outside. In an exemplary embodiment, when the isolation unit 400 and the placement unit 200 are coupled to each other by relative movement of the isolation unit 400 and the placement unit 200, the isolation space may be formed according to this coupling. By way of example, and not limitation, the isolation space may include a sealed space. In this example, the inside of the isolation space may be sealed from the external environment.

In an exemplary embodiment, the main body 100 may be located behind the upper member 300 and the placement unit 200. As an example, the main body 100 and the upper member 300 and/or the main body 100 and the placement unit 200 may be formed as one piece. As another example, the main body 100 and the upper member 300 and/or the main body 100 and the placement unit 200 may be configured to be coupled to each other.

In an exemplary embodiment, a power supply unit 530, an exhaust pump 630, and a filter 660 may be provided inside the main body 100.

In an exemplary embodiment, a chamber 110 may be provided at an upper part of the main body 100. In the chamber 110, an accommodation space in which at least one of the object to be treated (IM), the gripping device 20 for gripping the object to be treated (IM), and/or the container (not shown) for storing the object to be treated (IM) can be accommodated may be formed. A chamber door 120 capable of opening and closing the chamber 110 may be provided on one surface of the chamber 110, so that when the chamber door 120 is in an open state, the accommodation space inside the chamber 110 may be exposed to the outside. When the chamber door 120 is located in an open state, at least one of the object to be treated (IM), the gripping device 20 for gripping the object to be treated (IM), and/or the container (not shown) for storing the object to be treated (IM) may be accommodated in the accommodation space inside the chamber 110. When the chamber door 120 is in a closed state, the accommodation space inside the chamber 110 and the external environment may be blocked. As an example, when the chamber door 120 is in a closed state, the accommodation space inside the chamber 110 may be configured as an isolation space. By way of example, and not limitation, the chamber door 120 may be moved between an open position and a closed position by hinge movement relative to a hinge. As another example, the chamber door 120 may be moved in a sliding manner to achieve an open or closed state.

In an exemplary embodiment, a first UV light source unit 130 that irradiates the inside of the chamber 110 with ultraviolet (UV) may be provided inside the chamber 110. By way of example, and not limitation, the first UV light source unit 130 may include an LED capable of irradiating ultraviolet (UV) light. The first UV light source unit 130 may sterilize at least one of the object to be treated (IM), the gripping device 20 for gripping the object to be treated (IM), and/or the container (not shown) storing the object to be treated (IM) by irradiating at least one of the object to be treated (IM), the gripping device 20 for gripping the object to be treated (IM), and/or the container (not shown) for storing the object to be treated (IM) with ultraviolet (UV).

In an exemplary embodiment, an operation of taking out the object to be treated (IM) from the packaging by using the gripping device 20 may be performed inside the chamber 110. Accordingly, the object to be treated (IM) can be prevented from being contaminated by the external environment or the user's hand.

In an exemplary embodiment, the placement unit 200 may be located on one surface (for example, a front surface) of the main body 100. The placement unit 200 may be located in a direction opposite to the upper member 300. For example, the placement unit 200 may be located below the upper member 300.

In an exemplary embodiment, the upper surface of the placement unit 200 may be provided with a coupling member 210 into which a lower part of at least one of the object to be treated (IM), the gripping device 20 for gripping the object to be treated (IM), and/or the container (not shown) for storing the object to be treated (IM) is inserted. At least one of the object to be treated (IM), the gripping device 20 for gripping the object to be treated (IM), and/or the container (not shown) for storing the object to be treated (IM) connected to the placement unit 200 via the coupling member 210 may be fixed to the placement unit 220. Due to this fixation, the lower part of at least one of the object to be treated (IM), the gripping device 20 for gripping the object to be treated (IM), and/or the container (not shown) for storing the object to be treated (IM) can be more firmly coupled to a bottom surface 211 of the coupling member 210. Additionally, the placement unit 200 may be provided with a magnet 220. The magnet 220 may be formed on the bottom surface 211 of the coupling member 210. At least one of the object to be treated (IM), the gripping device 20 for gripping the object to be treated (IM), and/or the container (not shown) for storing the object to be treated (IM) may be coupled with the coupling member 210 more easily and more firmly by the magnet 220. In this regard, the object to be treated (IM), the gripping device 20 for gripping the object to be treated (IM), and/or the container (not shown) for storing the object to be treated (IM) that can be coupled with the coupling member 210 may include a metal material in at least a part thereof, making it possible to facilitate the coupling with the magnet 220. Accordingly, when at least one of the object to be treated (IM), the gripping device 20 for gripping the object to be treated (IM), and/or the container (not shown) for storing the object to be treated (IM) is inserted into the coupling member 210, the lower part thereof may be coupled to the bottom surface 211 of the coupling member 210 by the magnet 220. The apparatus 10 according to an exemplary embodiment of the present disclosure is provided with a magnet 220, making it possible to further facilitate the coupling between the placement unit 200 and at least one of the object to be treated (IM), the gripping device 20 for gripping the object to be treated (IM), and/or the container (not shown) for storing the object to be treated (IM). Such easiness of the coupling makes it possible to increase the convenience of a user who performs plasma surface treatment and the user experience.

In an exemplary embodiment, the coupling member 210 present on the upper part of the placement unit 200 may have various shapes depending on aspects of implementation. Examples of such a coupling member may include a coupling member using a hole, a coupling member using a magnet, a coupling member using a protrusion, a coupling member using an adhesive material, a coupling member using a belt, and/or a coupling member using tongs.

In an exemplary embodiment, the isolation unit 400 may move relative to the placement unit 200 to seal the object to be treated (IM) from the external environment. The relative movement of the isolation unit 400 and the placement unit 200 may include at least one of, for example, a first movement method in which the isolation unit 400 moves in a direction of the placement unit 200, a second movement method in which the placement unit 200 moves in a direction of the isolation unit 400, and a third movement method in which the isolation unit 400 and the placement unit 200 move toward each other. The isolation unit 400 can form along with the placement unit 200 an isolation space in which an inside is sealed against the external environment by moving relative to the placement unit 200. The isolation unit 400 and the placement unit 200 may move relative to each other and come into contact with each other to form an isolation space with a sealed inside. For example, as at least one of the isolation unit 400 and the placement unit 200 is raised and lowered, one surface of the isolation unit 400 comes into contact with one surface of the placement unit 200, so that an isolation space may be formed inside the isolation unit 400. The isolation unit 400 is connected to the elevating part 310 provided in the upper member 300 and can perform an elevating operation.

In an exemplary embodiment, the upper member 300 may accommodate at least a part (e.g., all) of the isolation unit 400. The isolation unit 400 can move up and down between a sealed position in which the isolation unit is in contact with the placement unit 200 and an accommodation position in which the isolation unit is accommodated inside the upper member 300.

In an exemplary embodiment, a hollow portion 410 may be formed at one location (for example, a center) of the isolation unit 400. Accordingly, the isolation unit 400 may be configured as a dual structure including an inner wall 420 and an outer wall 430.

When the isolation unit 400 moves and the lower surface of the isolation unit 400 comes into contact with the upper surface of the placement unit 200, the hollow portion 410 of the isolation unit 400 may form an isolation space. Since at least one of the object to be treated (IM) placed on the placement unit 200, the gripping device 20 for gripping the object to be treated (IM), and/or the container (not shown) for storing the object to be treated (IM) is located inside the hollow portion 410 of the isolation unit 400, when the isolation unit 400 and the placement unit 200 are in contact with each other to form an isolation space, at least one of the object to be treated (IM), the gripping device 20 for gripping the object to be treated (IM) and/or the container (not shown) for storing the object to be treated (IM) is located in the isolation space.

In an exemplary embodiment, the inner surface of the inner wall 420 may correspond to an inner surface of the hollow portion 410, and the outer surface of the outer wall 430 may correspond to an outer surface of the isolation unit 400.

In an exemplary embodiment, a second electrode 520 may be located between the inner wall 420 and the outer wall 430. As an example, the second electrode 520 may be formed in a manner of being coated on an outer surface of the inner wall 20 and an inner surface of the outer wall 430. The second electrode 520 may include a transparent electrode. The second electrode 520 may be configured to provide electrical conductivity to the isolation unit 400.

In an exemplary embodiment, a coating layer (not shown) may be formed on the inner surface of the inner wall 420. The coating layer may be made of a material that can withstand high temperatures and high voltages in order to prevent the inner surface of the inner wall 420 from being damaged or foreign matters from being eluted due to high temperatures and high voltages during plasma discharge. For example, the coating layer may be made of a heat-resistant material and/or an insulating material.

In an exemplary embodiment, the coating layer may include a material containing calcium. As such, when the coating layer is made of a material containing calcium, calcium can be allowed to adhere to the surface of the object to be treated (IM) by inducing elution of calcium by plasma discharge. In this way, by artificially causing calcium to adhere to the surface of the object to be treated (IM), when implanting or engrafting the object to be treated (IM) in the human body, the inflammatory response can be suppressed or alleviated, and furthermore, the implantation or engraftment is allowed to be more robust, ensuring a high implantation rate and/or a high engraftment rate.

In an exemplary embodiment, the coating layer may include a biocompatible material. Since the biocompatible material may adhere to the surface of the object to be treated (IM) during a plasma treatment process, the object to be treated (IM) can be more stably implanted or engrafted in the human body.

In an exemplary embodiment, an inner side of the isolation unit 400 may be made of a chemical-resistant material or formed with a chemical-resistant coating layer. At least a part of the isolation unit 400 is made of a transparent material to allow the atmosphere under a lower pressure state discharged inside the isolation unit 400 to be confirmed with a naked eye from the outside. The transparent material may include, for example, a glass material. By way of example, and not limitation, the inner wall 420 and the outer wall 430 of the isolation unit 400 may have a shape of a tubular tube made of transparent tempered glass. The isolation unit 400 may include a material having elasticity that is deformed so that the inside of the isolation unit 400 is sealed against the external environment using a pressure difference between the inside and the outside of the isolation unit 400.

In an exemplary embodiment, the isolation unit 400 may be connected to the elevating part 310. Therefore, the isolation unit 400 may be raised or lowered as the elevating part 310 is raised or lowered.

In an exemplary embodiment, an elevating part 310 may be provided inside the upper member 300. The elevating part 310 may be connected to a shaft 330 by a connecting member 320. The shaft 330 can be rotated by the motor 340. The connecting member 320 and the elevating part 310 can be raised or lowered by rotation of the shaft 330. Therefore, the shaft 330 can be rotated by driving of the motor 340, which can raise or lower the connecting member 320 and the elevating part 310. As such, the isolation unit 400 may be positioned to correspond to positions of the connecting member 320 and the elevating part 310. For example, when the connecting member 320 and the elevating part 310 are at raised positions, the isolation unit 400 is also located at a raised position, and accordingly, most or all of the region of the isolation unit 400 can be inserted into the inside of the upper member 300. When the connecting member 320 and the elevating part 310 are at lowered positions, the isolation unit 400 is also located at a lowered position, and accordingly, the isolation unit 400 can be taken out to the outside of the upper member 300 and come into contact with the upper surface of the placement unit 200.

In an exemplary embodiment, an elastic member 350 may be provided between the elevating part 310 and the isolation unit 400. As an example, one or more elastic members 350 may be present in an upper part of the isolation unit 400. The isolation unit 400 may include an elastic member 350 that is deformed so that the inside of the isolation unit 400 is sealed against the external environment by using a pressure difference between the inside and the outside of the isolation unit 400. The elastic member 350 can alleviate impact that is transmitted to the elevating part 310 and the isolation unit 400 when the elevating part 310 and the isolation unit 400 are lowered and, accordingly, the lower surface of the isolation unit 400 comes into contact with the upper surface of the placement unit 200. The elastic member 350 can make the seal between the isolation unit 400 and the placement unit 200 more robust. The elastic member 350 can further improve the sealing force between the lower surface of the isolation unit 400 and the upper surface of the placement unit 200 by generating elastic force in the process of contact between the isolation unit 400 and the placement unit 200.

In an exemplary embodiment, the remaining region of the region corresponding to the placement unit 200, excluding the coupling member 210 that allows electrical connection, may be composed of the elastic member 350.

In an exemplary embodiment, a second UV light source unit 360 may be provided to the elevating part 310. The second UV light source unit 360 may irradiate the inside of the isolation unit 400 with ultraviolet (UV). The second UV light source unit 360 may irradiate the isolation space with ultraviolet (UV). The second UV light source unit 360 may irradiate UV downward while coupled to the elevating part 310. As the second UV light source unit 360 irradiates ultraviolet (UV) into the isolation unit 400, the internal space of the isolation unit 400 may be sterilized. Accordingly, when the object to be treated (IM) is located inside the isolation unit 400, the object to be treated (IM) can be prevented from being contaminated. For example, the second UV light source unit 360 may include an LED capable of irradiating ultraviolet (UV) light. In an exemplary embodiment, the second UV light source unit 360 may irradiate ultraviolet (UV) while fixedly coupled to the elevating part 310, or the second UV light source unit 360 may direct ultraviolet (UV) to various positions inside the isolation space by moving relative to the elevating part 310 or rotating.

In an exemplary embodiment, when the placement unit 200 and the isolation unit 400 are sealed as the isolation unit 400 and the placement unit 200 move relative to each other, the treatment unit 500 may perform or control plasma treatment by discharging plasma inside the hollow portion 410 of the isolation unit 400 forming the isolation space.

In an exemplary embodiment, the treatment unit 500 may be included inside the main body 100. The treatment unit 500 may include a first electrode 510 provided in the placement unit 200 to be electrically connected to the object to be treated (IM), a second electrode 520 provided in the isolation unit 400 to surround the object to be treated (IM), a power supply unit 530 to apply power to the first electrode 510 and the second electrode 520, a first conducting wire 540 connecting the first electrode 510 and the power supply unit 530, and a second conducting wire 550 connecting the second electrode 520 and the power supply unit 530. As an example, the power supply unit 530 may form an electric field during a process time for which an internal pressure of the isolation unit 400 is within a preset process pressure range, thereby generating alternating current power (AC) for discharging the atmosphere under a lower pressure state.

A plasma reaction in the present disclosure may encompass a plasma reaction generated by applying a direct current voltage, a plasma reaction generated by applying RF or microwave, and/or an inductively coupled plasma (ICP) reaction, and the like.

In an exemplary embodiment, the first electrode 510 may be provided in the bottom surface 211 of the coupling member 210 of the placement unit 200. When the gripping device 20 is placed while being coupled (for example, inserted) to the coupling member 210 of the placement unit 200, a lower surface of the gripping device 20 comes into contact with the first electrode 510. Portions where the gripping device 20 and the object to be treated (IM) are connected may be made of a metal material so that the gripping device 20 and the object to be treated (IM) are electrically connected to each other. Therefore, when the gripping device 20 is placed on the placement unit 200, the first electrode 510 connected to the placement unit 200, the gripping device 20 and the object to be treated (IM) may be electrically connected.

In an exemplary embodiment, at least a part of the first electrode 510 may be exposed to the inside of the isolation space. The first electrode 510 is electrically connected to the object to be treated (IM) or at least a part of the container in which the object to be treated (IM) is stored, which are present in the isolation space, so that the efficiency of the plasma reaction to the object to be treated (IM) can be increased.

In an exemplary embodiment, the second electrode 520 may be provided between the inner wall 420 and the outer wall 430 of the isolation unit 400. When the isolation unit 400 and the placement unit 200 form an isolation space, the second electrode 520 may be formed or disposed to correspond to a shape of the object to be treated (IM). As an example, when an isolation space is formed, the second electrode 520 may be configured to surround the object to be treated (IM).

In an exemplary embodiment, the first conducting wire 540 may be disposed inside both the placement unit 200 and the main body 100. The second conducting wire 550 may be disposed inside both the upper member 300 and the main body 100. The first conducting wire 540 may be disposed along the lower part of the apparatus 10 for plasma surface treatment, and the second conducting wire 550 may be disposed along the upper part of the apparatus 10 for plasma surface treatment.

Additionally, the second conducting wire 550 may also be disposed in the placement unit 200. In this case, the second conducting wire 550 may be disposed inside both the placement unit 200 and the main body 100. Accordingly, a ground portion (not shown) may be connected to an end portion of the second conducting wire 550, and the ground portion may be located at a position corresponding to the second electrode 520 on the upper surface of the placement unit 200. In this way, in the case where the second conducting wire 550 is provided in the placement unit 200, when the isolation unit 400 is in the raised state, the second electrode 520 and the second conducting wire 550 are not electrically connected to each other. When the isolation unit 400 is lowered and thus the second electrode 520 and the ground portion come into contact with each other, the second electrode 520 is electrically connected to the second conducting wire 550, so that the power supply unit 530 and the second electrode 520 are electrically connected. When the second electrode 520 of the isolation unit 400 and the ground portion of the placement unit 200 come into contact with each other and the object to be treated (IM) is sealed from the external environment, the second electrode 520 may be electrically connected to the power supply unit 530.

The description of the arrangement of the first conducting wire 540 and the second conductor 500 is provided as an example for the purpose of explanation, and depending on the implementation aspect, the first conducting wire 540 and the second conducting wire 550 may be disposed at any positions in the main body 100 where the first electrode 510 and the power supply unit 530 and the second electrode 520 and the power supply unit 530 can be connected.

In an exemplary embodiment, the inner wall 420 of the isolation unit 400 may be made of a dielectric barrier layer. When the isolation unit 400 and the placement unit 200 are in contact with each other and sealed to form an isolation space, the power supply unit 530 can apply power to the first electrode 510 and the second electrode 520. Accordingly, plasma discharge may occur inside the hollow portion 410 of the isolation unit 400 forming the isolation space. Plasma discharge may occur inside the dielectric barrier of the isolation unit 400 forming the isolation space. When plasma is generated inside the isolation space, the surface of the object to be treated (IM) is modified from hydrophobicity to hydrophilicity, so that plasma surface treatment can be performed.

In an exemplary embodiment, the exhaust unit 600 may exhaust the inside of the isolation unit 400. The exhaust unit 600 may exhaust the atmosphere inside the isolation unit 400. The exhaust unit 600 may exhaust the atmosphere inside the isolation unit 400 to make the isolation space into a low-pressure atmospheric state or a vacuum state. The exhaust unit 600 may adjust the internal pressure of the isolation unit 400 to be within a preset process pressure range. By way of example, and not limitation, the exhaust unit 600 may include any type of member capable of communicating the atmosphere, and as an example, the exhaust unit 600 may include a mechanical or electric air intake for sucking air. By way of example, and not limitation, the preset process pressure range may be a range of 0.001 Torr or higher and lower than 100 Torr. As another example, the preset process pressure range may be a range of 1 Torr or higher and lower than 30 Torr.

FIG. 5C is a perspective view illustratively showing a state in which the isolation unit 400 is lowered to seal the object to be treated (IM) from the external environment according to the exemplary embodiment of the present disclosure. FIG. 5D is a cross-sectional view illustratively showing a state in which the isolation unit 400 is lowered to seal the object to be treated (IM) from the external environment. FIG. 5E is a perspective view illustratively showing the inside of the apparatus 10 for plasma surface treatment according to the exemplary embodiment of the present disclosure.

In an exemplary embodiment, the exhaust unit 600 may serve to exhaust air inside the isolation unit 400 sealed from the external environment. The exhaust unit 600 may include an exhaust port 610 provided in the placement unit 200, an exhaust flow passage 620 that communicates with the exhaust port 610, an exhaust pump 630 that communicates with the exhaust flow passage 620 to generate suction force, a pump valve 640 that interrupts communication between the exhaust pump 630 and the exhaust flow passage 620, a venting valve 650 provided on the exhaust flow passage 620, a filter 660 that filters out foreign matters in the exhaust flow passage, and/or a pressure sensor 670 that measures a pressure in the exhaust flow passage 620. The pressure sensor 670 may measure a pressure of the isolation unit 400 and/or the exhaust flow passage 620. By way of example, and not limitation, the pressure sensor 670 may include any type of device that outputs a magnitude of fluid pressure applied to the pressure sensor 670 as an electrical signal. The pressure sensor 670 may output a pressure in pascal units needed to prevent a fluid from expanding and measure an absolute pressure, a gauge pressure, and/or a differential pressure.

In an exemplary embodiment, the exhaust port 610 may be provided in the coupling member 210 of the placement unit 200. A hole is formed in at least a part of the coupling member 210 or the placement unit 200. Thus, when the isolation unit 400 forms an isolation space, air inside the isolation unit 400 may be exhausted to the outside of the isolation unit 400 through the exhaust port 610.

In an exemplary embodiment, the exhaust flow passage 620 is a path that communicates the exhaust port 610 and the exhaust pump 630.

In an exemplary embodiment, the exhaust pump 630 may be used to transfer gas from a specific space to another space. The exhaust pump 630 may be provided on the exhaust flow passage 620 to exhaust the air inside the isolation unit 400 to the outside of the isolation space upon actuation. For example, the exhaust pump 630 may continuously perform an exhaust operation to maintain the internal pressure of the isolation unit 400 constant (e.g., in a low-pressure state), and plasma discharge may be performed in the isolation space of the isolation unit 400 maintained in the low-pressure state. As another example, the exhaust pump 630 may perform an exhaust operation for varying the internal pressure of the isolation unit 400, and accordingly, the plasma discharge may be performed in the isolation space of the isolation unit 400 while the atmospheric pressure is varied.

In an exemplary embodiment, the pump valve 640 may be provided on the exhaust flow passage 620 and can perform opening and closing operations. When the pump valve 640 is opened, the exhaust pump 630 and the exhaust flow passage 620 can be communicated. When the pump valve 640 is closed, communication between the exhaust pump 630 and the exhaust flow passage 620 can be interrupted. Therefore, when the exhaust pump 630 is actuated with the pump valve 640 open, the air inside the isolation unit 400 can be exhausted, and when the pump valve 540 is closed, the air inside the isolation unit 400 is not exhausted even if the exhaust pump 630 is actuated. By way of example, and not limitation, the pump valve 640 may include a solenoid valve whose opening and closing operations can be performed by a controller (not shown). For example, the internal pressure of the isolation unit 400 may be maintained constant according to the opening and closing operations of the pump valve 640, and plasma discharge may be performed inside the isolation unit 400 in which the pressure of the atmosphere under a lower pressure state is maintained constant. As another example, the internal pressure of the isolation unit 400 may vary according to the opening and closing operations of the pump valve 640, and accordingly, plasma discharge may be performed in the isolation space of the isolation unit 400 while the atmospheric pressure is varied.

In an exemplary embodiment, the venting valve 650 may be provided to be opened and closed on the exhaust flow passage 620. When the venting valve 650 is opened, the exhaust flow passage 620 is opened and the air inside the isolation unit 400 can be exhausted. When the venting valve 650 is closed, the exhaust flow passage 620 is closed and the air inside the isolation unit 400 is not exhausted. The venting valve 650 can stop the exhaust of air inside the isolation unit 400 by exhausting the inside of the isolation unit 400 or interrupting the exhaust flow passage 620 during the plasma treatment process according to the opening or closing operation.

In an exemplary embodiment, the controller may use the pressure sensor 670 and the exhaust pump 630 to put the inside of the isolation unit 400 into a preset pressure state.

In an exemplary embodiment, the exhaust unit 600 may allow the treatment unit 500 to exhaust the air inside the isolation unit 400 so that plasma treatment can be performed in the isolation space under a vacuum state or a low-pressure atmosphere state.

According to an exemplary embodiment of the present disclosure, since the isolation unit 400 and the placement unit 200 form an isolation space by moving relative to each other, plasma surface treatment for the object to be treated (IM) can be performed in a space sealed from the external environment.

According to an exemplary embodiment of the present disclosure, since the plasma treatment can be performed while the inside of the isolation unit 400 is exhausted by the exhaust unit 600, foreign matters such as hydrocarbons on the surface of the object to be treated (IM) can be effectively removed. Accordingly, the hydrophilicity of the surface of the object to be treated (IM) can be improved.

According to an exemplary embodiment of the present disclosure, after the exhaust operation by the exhaust unit 600 is performed during the plasma operation, the inside of the isolation unit 400 can be vented through the venting valve 650. As a result, an ozone concentration inside the isolation unit 400 increases, so that a high sterilization effect on the surface of the object to be treated (IM) can be achieved.

According to an exemplary embodiment of the present disclosure, sterilization through ultraviolet and surface modification through plasma can be performed together, so that a high level of surface treatment for the object to be treated (IM) can be achieved.

According to an exemplary embodiment of the present disclosure, when the isolation unit 400 is exhausted again after the plasma treatment is completed, foreign matters generated during the plasma treatment process can be removed, so that foreign matters on the surface of the object to be treated (IM) can be prevented from being re-deposited, and further, a high-level of surface treatment for the object to be treated (IM) can be ensured.

According to an exemplary embodiment of the present disclosure, the gripping device 20, the object to be treated (IM), and/or the container can be more firmly placed through the magnet 220 and the coupling member 210.

According to an exemplary embodiment of the present disclosure, the isolation unit 400 and/or the second electrode 520 may be made of a transparent material, so that the plasma treatment process for the object to be treated (IM) can be confirmed with a naked eye from the outside. Accordingly, a more reliable user experience for the plasma treatment process can be achieved.

FIG. 5F is a perspective view illustratively showing an open state of a chamber 110 of the apparatus 10 for plasma surface treatment according to the exemplary embodiment of the present disclosure.

As illustrated in FIG. 5F, one surface of the chamber 110 may include a chamber door 120. The chamber door 120 can be moved between an open position and a closed position. When the chamber door 120 is at the open position, a space inside the chamber 110 may be exposed to the outside. When the chamber door 120 is at the closed position, the space inside the chamber 110 may be blocked (for example, sealed) from the outside. As shown in FIG. 5, the chamber door 120 can be moved between the open position and the closed position through hinge movement. As another example, the chamber door 120 may open or close the chamber 110 through a sliding operation. As another example, the chamber door 120 can be separated from and coupled to the chamber 110, so that when separated, the chamber door can open the inside of the chamber 110, and when coupled, the chamber door can close the inside of the chamber 110.

In an exemplary embodiment, at least one of the object to be treated (IM), the gripping device 20 for gripping the object to be treated (IM), and the container for storing the object to be treated (IM) may be placed on a part of the chamber door 120. Accordingly, the chamber door 120 and the object to be treated (IM) can be connected in a state in which the chamber door 120 is in the open position, and the chamber door 120 can be moved to the closed position in the state in which the chamber door 120 and the object to be treated (IM) are connected. As the chamber door 120 is moved to the closed position, the object to be treated (IM) may be present in the chamber 110 while being blocked from the external environment.

In an exemplary embodiment, sterilization treatment for the object to be treated (IM) using ultraviolet (UV) may be performed inside the chamber 110. In an exemplary embodiment, plasma surface treatment may be performed for the object to be treated (IM) inside the chamber 110.

As illustrated in FIG. 5F, a plurality of reactions to the object to be treated (IM) may occur inside the chamber 110. The technique according to an exemplary embodiment of the present disclosure can perform parallel plasma treatments for a plurality of objects (IM) to be treated together with the object to be treated (IM) placed on the placement unit 200.

In an exemplary embodiment, at least one surface of the chamber door 120 may include a light-transmissive member. The light-transmissive member may be configured such that the at least one surface allows transmission of visible light from the inside of the chamber 110 to the outside during at least one section of the operation period of the apparatus 10. At least a part of the chamber door 120 may be configured to allow transmission of visible light corresponding to a wavelength range of a region that reacts with plasma inside the chamber 110, during at least one section of the operation period of the apparatus 10.

The light-transmissive member in the present disclosure may include transparent glass, transparent plastic, and/or transparent conductive material.

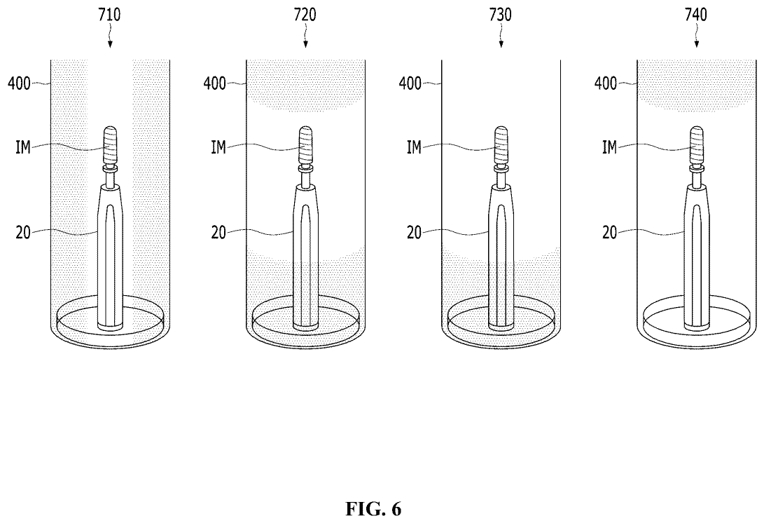

FIG. 6 illustratively shows the isolation unit 400 having at least one surface including a light-transmissive member according to various embodiments of the present disclosure.

In an exemplary embodiment, at least one surface of the isolation unit 400, which together with the placement unit 200 forms an isolation space in which an inside is sealed against the external environment during the operation period of the apparatus 10, may include a light-transmissive member.

In an exemplary embodiment, the isolation unit 400 may be configured such that least one surface allows transmission of visible light from the isolation space to the outside during at least one section of the operation period of the apparatus 10. For example, at least one surface of the isolation unit 400 may include a transparent material. Examples of the transparent material may include transparent plastic including transparent acryl and transparent polycarbonate, transparent glass, transparent ceramic, and/or transparent conductive material.

In an exemplary embodiment, the isolation unit 400 may be configured to allow transmission of visible light corresponding to a wavelength range of a region that reacts with plasma in the isolation space, during at least one section of the period of the apparatus 10. In this example, at least one surface of the isolation unit 400 may include a material that can transmit light within a wavelength range of visible light (for example, a wavelength of 590 to 750 nm) generated resulting from a plasma reaction. By way of example, and not limitation, the above-described material may include a film having a filter function of transmitting light within a specific wavelength range and blocking light within other wavelength ranges, or the like. In this example, when the film having the above-described function is attached or applied to the outer surface of the isolation unit 400, a function that can transmits light of a specific wavelength may be implemented. The above-described wavelength range is described for illustrative purposes, and depending on the implementation aspect, a wavelength range of any visible light that can appear resulting from the plasma reaction may fall within the scope of the present disclosure.

In an exemplary embodiment, at least one surface of the isolation unit 400 may include a light-transmissive member that can block visible light within a specific wavelength range and can transmit visible light within other specific wavelength ranges. For example, a transparent member may form an outer peripheral surface of the isolation unit 400, and a light-transmissive member that transmits light within a wavelength range that reacts with plasma may be attached to the transparent member. Depending on the implementation aspect, a light-transmissive member or a transparent member may be disposed at a predetermined position on the outer peripheral surface of the isolation unit 400. As another example, a light-transmissive member itself that transmits light within a wavelength range that reacts with plasma may form the outer surface of the isolation unit 400.

Exemplary embodiments of the present disclosure illustrated in FIG. 6 illustratively show various arrangements of the light-transmissive member in the isolation unit 400 depending on implementation aspects. An exemplary embodiment indicated by reference numeral 710 represents a light-transmissive member formed along a longitudinal direction of the isolation unit 400. An exemplary embodiment indicated by reference numeral 720 represents a light-transmissive member formed along a transverse direction of the isolation unit 400. An exemplary embodiment indicated by reference numeral 730 represents a light-transmissive member occupying the upper part of the isolation unit 400. An exemplary embodiment indicated by reference numeral 740 represents a light-transmissive member occupying the lower part of the isolation unit 400.

The exemplary embodiments shown in FIG. 6 represent some of the various exemplary embodiments falling within the scope of the present disclosure. Although not shown in FIG. 6, an exemplary embodiment in which a light-transmissive member occupies the entire isolation unit 400 and/or an exemplary embodiment in which a light-transmissive member occupies only a specific portion of the outer surface of the isolation unit 400 (for example, a light-transmissive member occupies a position corresponding to a direction in which a user who uses the apparatus 10 is looking) may also fall within the scope of the present disclosure.

In an exemplary embodiment, a region of the isolation unit 400 that is not occupied by the light-transmissive member may be made of, for example, an opaque material or may include a material that blocks light transmission.

In an exemplary embodiment, the operating period of apparatus 10 in the present disclosure may be divided into a plurality of sections. By way of example, and not limitation, the operation period of the apparatus 10 may include a first section during which the object to be treated (IM) and the placement unit 200 are connected to each other, a second section during which an isolation space that blocks the object to be treated (IM) from the external environment is formed by relative movement of the isolation unit 400 and the placement unit 200, a third section during which plasma surface treatment is performed for the object to be treated (IM) by forming an electric field in the isolation space in a state in which the isolation space is formed, a fourth section during which the object to be treated (IM) is present inside the isolation space after the plasma surface treatment is completed, and/or a fifth section during which the isolation space is released by relative movement of the isolation unit 400 and the placement unit 200 and thus the object to be treated (IM) is exposed to the external environment.

In another exemplary embodiment, the operation period of the apparatus 10 in the present disclosure may be divided into a plurality of sections. By way of example, and not limitation, the operation period of the apparatus 10 may include a first section during which a current state of the apparatus 10 is checked, a second section during which an isolation space that blocks the object to be treated (IM) from the external environment is generated as the isolation unit 400 is lowered and coupled with the placement unit 200, a third section during which a pump operation is performed, a fourth section during which plasma surface treatment is performed for the object to be treated (IM) as a high voltage is applied, a fifth section during which an exhaust or pump operation is performed to remove impurities and to perform a cooling operation after plasma surface treatment, and a sixth section during which the isolation unit 400 is moved up and a cleaning operation is performed based on the exhaust or pump operation.

In an exemplary embodiment, the transmission of visible light from the isolation unit 400 to the outside and/or the transmission of visible light corresponding to a wavelength range of a region that reacts with plasma may occur during a specific section or multiple sections among the plurality of sections of the operation period of the apparatus 10.

In such a case, the controller (not shown) may control the isolation unit 400 to adjust transparency inside the isolation space and/or visible light transmittance. In an exemplary embodiment, the isolation unit 400 may include a material capable of adjusting visible light transmittance, such as an active optical film. As an example, the visible light transmittance of the isolation unit 400 is adjusted during a section during which plasma surface treatment is performed, so that the plasma treatment process can be seen from the outside. In this example, the visible light transmittance of the isolation unit 400 may be adjusted during sections other than the section during which plasma surface treatment is performed so that the inside of the isolation space is not seen from the outside. In this way, each of the plurality of sections of the operation period of the apparatus 10 and the visible light transmittance of the outer surface of the isolation unit 400 can be set to have a correlation with each other.

Additionally, the outer surface of the isolation unit 400 may include any material that can adjust light transmittance (for example, visible light transmittance) by applying light or an electric field. As an example, the isolation unit 400 may adjust the light transmittance by applying an electric field from the power supply unit 530.

As described above, the apparatus 10 according to an exemplary embodiment of the present disclosure can visually expose the plasma treatment process to the outside while maintaining a vacuum state or low-pressure state during the plasma treatment process. Accordingly, not only the quality of the surface treatment of the object to be treated (IM) according to the plasma treatment process but also the reliability of the plasma treatment process provided to the user can be guaranteed. Accordingly, the user experience of the user who performs plasma treatment for the object to be treated (IM) with the apparatus 10 can be increased.

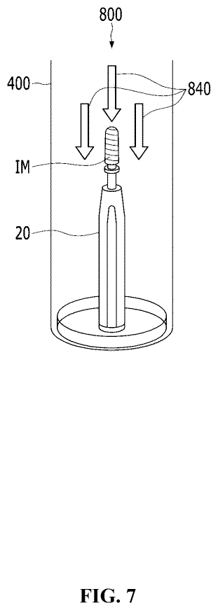

FIG. 7 illustratively shows a direction 840 in which plasma discharge occurs as an electric field is formed in an isolation space according to an exemplary embodiment of the present disclosure.

The direction 840 shown through reference numeral 800 in FIG. 7 is expressed for illustrative purposes. As illustrated in FIG. 7, plasma discharge with various directionality depending on implementation aspect such as a lateral direction or a circular direction other than the longitudinal direction 840 is also possible.

In an exemplary embodiment, a discharge direction or discharge path of plasma may be determined by a structure of the exhaust unit 600 connected to the isolation space, a dielectric structure inside the isolation space, and a structure of the electrode (510 and/or 520) formed inside the isolation space. As an example, a plasma reaction may occur along the direction 840 toward the first electrode 510 located proximal to the placement unit 200. As shown in FIG. 7, the object to be treated (IM) may sequentially react with plasma along the longitudinal direction 840 of the isolation space.

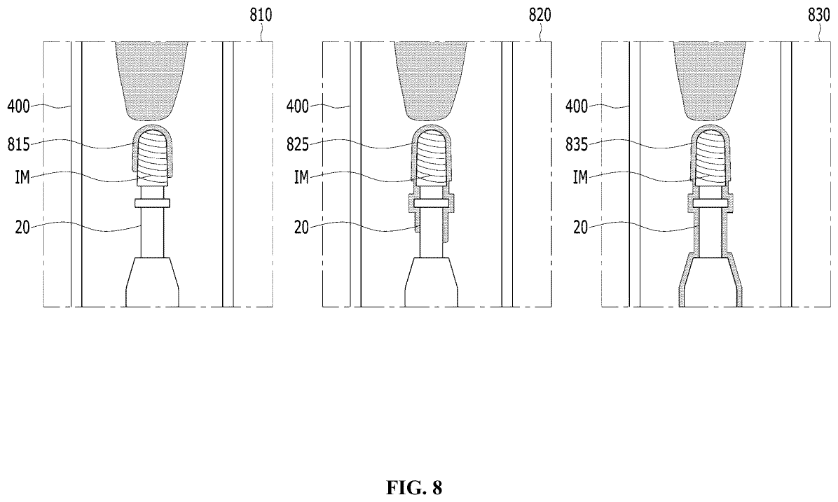

FIG. 8 illustratively shows a state in which a region reacting with plasma in the isolation space dynamically changes according to an exemplary embodiment of the present disclosure.

In an exemplary embodiment, during the operation period of the apparatus 10, a region that reacts with plasma in the isolation space may change dynamically. As an example, the region that reacts with plasma may correspond to the object to be treated (IM) and at least a part of the gripping device 20 that connects the object to be treated (IM) to the placement unit 200. In an exemplary embodiment, a region of the object to be treated (IM) that reacts with plasma within the isolation space may vary over time. As an example, the region of the object to be treated (IM) that reacts with plasma may increase over time. In this example, a reaction region at a first time point during the operation period of the apparatus 10 may be included in a reaction region at a second time point (a time point subsequent to the first time point) during the operation period of the apparatus 10. As another example, a region of the object to be treated that reacts with plasma may be different at the first time point and the second time point during the operation period of the apparatus 10. In this case, the plasma reaction region of the object to be treated (IM) at the first time point may not correspond to the plasma reaction region of the object to be treated (IM) at the second time point. Here, the first time point and the second time point represent temporally different time points.

As illustrated by reference numeral 810 in FIG. 8, a plasma reaction may occur on at least a part 815 of the object to be treated (IM) at the first point during the plasma treatment process. This plasma reaction can be visually confirmed from the outside. As illustrated by reference numeral 820, it can be visually confirmed that a plasma reaction occurs on the object to be treated (IM) and at least a part 825 of the gripping device 20 at the second time point after the first time point during the plasma treatment process. In the example indicated by reference numeral 820, it can be confirmed that the plasma reaction occurs on the outer surface of the object to be treated (IM) and at least a part of the gripping device 20, as shown by reference numeral 825. As illustrated by reference numeral 830, it can be visually confirmed that a plasma reaction occurs on at least a part 835 of the object to be treated (IM) at a third time point after the second time point during the plasma treatment process. In the example indicated by reference numeral 830, it can be confirmed that the plasma reaction occurs on the outer surface of the object to be treated (IM) and the outer surface of the gripping device 20, as shown by reference numeral 835.

In an exemplary embodiment, as illustrated in FIG. 8, the object to be treated (IM) may react sequentially with plasma along the longitudinal direction of the isolation space. Such a sequential plasma reaction can be seen to the outside through the light-transmissive member of the isolation unit 400.

In an exemplary embodiment, the reaction region of the object to be treated (IM), the gripping device 20, and/or the container, which reacts with the plasma in the isolation space, may be formed based on the structure of the exhaust unit 600 connected to the isolation space, the operation method of the exhaust unit 600, the dielectric structure inside the isolation space, and the structure and arrangement of the electrodes 510 and 620 formed inside the isolation space.

As illustrated in FIG. 8, the object to be treated (IM) may sequentially react with plasma in the direction of the first electrode 510 located proximal to the placement unit 200. Here, the proximity with respect to the placement unit 200 is used to express a position relatively close to the placement unit 200. For example, a position proximal to the placement unit 200 may include the lower part of the gripping device 20, the lower part of the container, or the lower part of the object to be treated (IM), which are close to the placement unit 200, and a position distal from the placement unit 200 may include the upper part of the gripping device 20, the upper part of the container, or the lower or upper part of the object to be treated (IM), which are distant from the placement unit 200. In an exemplary embodiment according to the present disclosure, a plasma discharge path may be formed in a direction toward the first electrode 510 exposed to the inside of the isolation space, and accordingly plasma discharge may occur from the upper region toward the lower region of the isolation unit 400. Accordingly, as shown in FIG. 8, plasma discharge may occur in a direction from the upper part toward the lower part of the object to be treated (IM), and such a plasma discharge process can be visually confirmed from the outside through the isolation unit 400.

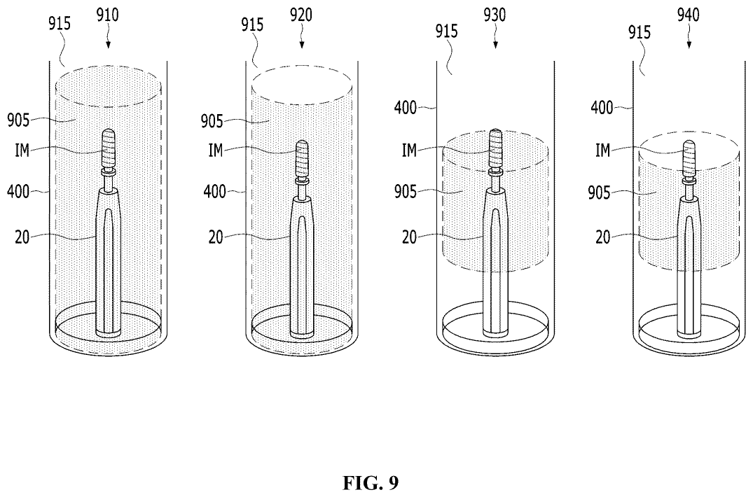

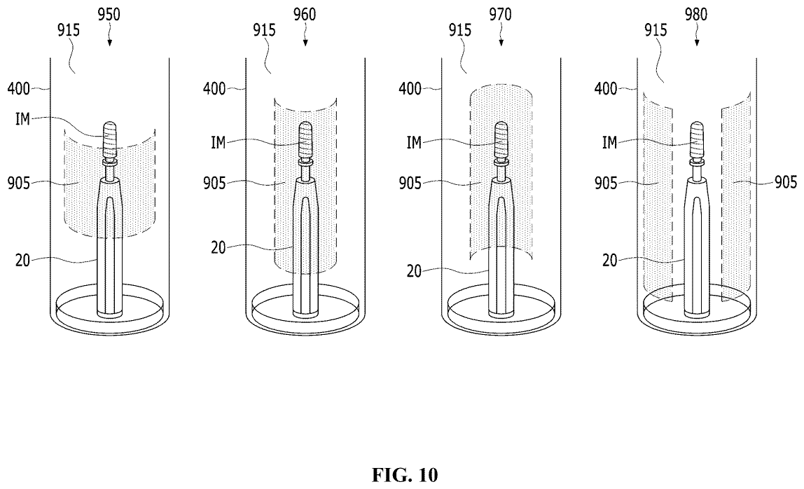

FIGS. 9 and 10 illustratively show a light-transmissive member 915 and a conductive member 905 in the isolation space according to various embodiments of the present disclosure. For illustrative purposes, regions that may be occupied by the conductive member 905 are shaded in a planar form in FIGS. 9 and 10. However, since the conductive member 905 can be implemented not only in the planar form but also in a point-like or line-like form, the conductive member 905 may occupy a partial region (for example, a line-like region or a point-like region) of the region shaded in a planar form in FIGS. 9 and 10 depending on these various implementation aspects.

In an exemplary embodiment, the isolation unit 400 may further include a conductive member 905 disposed to correspond to the light-transmissive member 915 in the isolation space. Here, the expression “A and B are disposed to correspond to each other” may mean that A and B are disposed in substantially the same region, that A and B are disposed parallel to each other, that A is disposed to occupy a part of B, that there is a correlation between positions or regions where A and B are disposed, and/or that at least a part of regions occupied by A and B overlaps.

In the present disclosure, the light-transmissive member 915 may include, for example, a fully transparent member, a partially transparent member, a member that transmits light within a specific wavelength range, and/or a member that can adjust light transmittance, as described above. In addition, the conductive member 905 may refer to a member that includes at least a part having low resistance against current circulation (i.e., having electrical conductivity). By way of example, and not limitation, the conductive member 905 may be made of a material such as metal, electrolyte, superconductor, semiconductor, graphite, and/or conductive polymer.

In an exemplary embodiment, at least one surface of the isolation unit 400 may include a light-transmissive and conductive member. A transparent electrode may be considered as an example of the light-transmissive and conductive member. The transparent electrode may be a thin transparent substrate that has both electric conductivity and light transmission properties. For example, a transparent conducting oxide (TCO) manufactured in the form of a thin film may be considered as a material for the transparent electrode. By way of example, and not limitation, a transparent conducting oxide may refer to a semiconductor material that simultaneously has high optical transmittance (85% or higher) in the visible light region and low resistivity (1×10⁻³ Ωcm). In an exemplary embodiment, the transparent electrode may correspond to the second electrode 520. Such a transparent electrode occupies at least a part of the inner surface of the isolation unit 400 and can be used to form an electric field for generating plasma together with the first electrode 510 exposed to the isolation space.

In an exemplary embodiment, the isolation unit 400 may include a conductive member 905 disposed along the longitudinal or transverse direction of the region occupied by the light-transmissive member 915 within the isolation space. The isolation unit 400 may include one or more conductive members 905.

In an exemplary embodiment, at least a part of the outer surface of the isolation space may include a transparent member 915 having light transmission properties, and at least a part of the inner surface of the isolation space may include a transparent electrode 905 having conductivity.

In an exemplary embodiment, since the conductive member 905 can operate as at least a part of the electrode part that forms an electric field inside the isolation unit 400, the plasma reaction inside the isolation space can be performed more effectively.

In an exemplary embodiment, in the isolation space indicated by reference numeral 910, the conductive member 905 may be provided in the isolation unit 400 in a shape corresponding to a shape of the light-transmissive member 915. Here, the conductive member 905 may occupy regions corresponding to the upper and lower surfaces inside the isolation space. For example, the upper and lower surfaces inside the isolation space may include two surfaces determined based on the longitudinal direction of the isolation space. Accordingly, not only a region that can be contacted with the first electrode 510 or the placement unit 200 but also a region at a position facing the first electrode 510 or the placement unit 200 may be occupied by the conductive member 905. As an example, the conductive member 905 may have a cylindrical shape, for example.

In an exemplary embodiment, in the isolation space indicated by reference numeral 920, the conductive member 905 may be provided in the isolation unit 400 in a shape corresponding to a shape of the light-transmissive member 915. In the example indicated by reference numeral 920, the conductive member 905 may occupy a cylindrical region with open upper and lower surfaces.

In an exemplary embodiment, in the isolation space indicated by reference numeral 930, the conductive member 905 may be provided in the isolation unit 400 in a shape corresponding to a shape of the light-transmissive member 915. In the example indicated by reference numeral 930, the conductive member 905 may occupy a cylindrical region with closed upper and lower surfaces. The example indicated by reference numeral 930 shows that the conductive member 905 is concentrated and formed at a position adjacent to the object to be treated (IM). A size of the occupied region of the conductive member 905 may be smaller than a size of the occupied region of the conductive member 905 in the isolation space indicated by reference numeral 910 or 920.

In an exemplary embodiment, in the isolation space indicated by reference numeral 940, the conductive member 905 may be provided in the isolation unit 400 in a shape corresponding to a shape of the light-transmissive member 915. In the example indicated by reference numeral 940, the conductive member 905 may occupy a cylindrical region with open upper and lower surfaces. The example indicated by reference numeral 940 shows a form in which the conductive member 905 is concentrated at a position adjacent to the object to be treated (IM). A size of the occupied region of the conductive member 905 may be smaller than a size of the occupied region of the conductive member 905 in the isolation space indicated by reference numeral 910 or 920.

In an exemplary embodiment, in the isolation spaces indicated by reference numerals 950, 960, and 970, the conductive member 905 may be provided in the isolation unit 400 in a shape corresponding to a shape of the light-transmissive member 915. In the examples indicated by reference numerals 950, 960, and 970, the conductive member 905 may occupy a partial region of the cylindrical light-transmissive member 915. Here, the partial region may include at least one of a front surface, a side surface, a rear surface, an upper surface, and a lower surface of the light-transmissive member 915. Additionally, the conductive member 905 may occupy a partial region of the light-transmissive member 915 along the longitudinal or transverse direction of the isolation space.

In an exemplary embodiment, a plurality of conductive members 905 may be disposed in the isolation space indicated by reference numeral 980. The number, arrangement position, and/or occupied region of the conductive members 905 may be determined in consideration of the plasma discharge path and the intensity of the plasma reaction to the object to be treated (IM).

As described above, the apparatus 10 according to an exemplary embodiment of the present disclosure has the configuration of the isolation unit 400 including the conductive member 905 and the light-transmissive member 915 and thus can improve the quality of plasma surface treatment for the object to be treated (IM) and increase the user experience by allowing the plasma surface treatment to be visually confirmed from the outside.

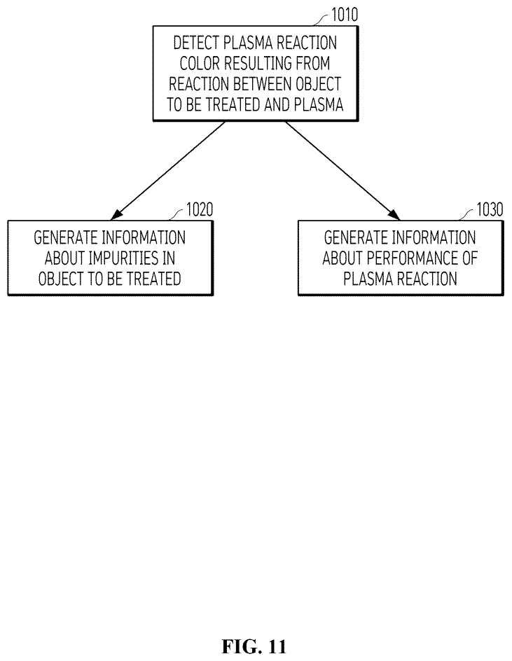

FIG. 11 illustratively shows an operation of a controller of the apparatus for plasma surface treatment according to an exemplary embodiment of the present disclosure.

In an exemplary embodiment, the apparatus 10 may further include a controller. Such a controller can control an overall operation of the apparatus 10 for plasma treatment.

In an exemplary embodiment, the controller refers to a computer-related entity, hardware, firmware, software, a combination of software and hardware, or execution of software, and may be used interchangeably. For example, a component may be, but is not limited to, a procedure running on a processor, a processor, an object, a thread of execution, a program, and/or a computer. In addition, the controller may provide appropriate information or functions to the user or process the same by processing data, information, signals and the like input or output through components included in the apparatus 10 or running an application program stored in a storage unit. Such a controller may consist of at least one core, and may include a processor for data analysis and/or processing, such as a central processing unit (CPU) of a computing device 100, a general purpose graphics processing unit (GPGPU), and a tensor processing unit. Additionally, the controller may include any type of computing system or computing apparatus, such as a microprocessor, a mainframe computer, a digital processor, a portable device, and a device controller.

In an exemplary embodiment, the controller may control the operation of the exhaust unit 600 so that the exhaust unit 600 can adjust the internal pressure of the isolation unit 400 to be within a preset process pressure range. In addition, the controller may control the exhaust unit 600 to form an atmosphere under a lower pressure state within a preset process pressure range inside the isolation unit 400, and control the operations of the power supply unit 530 and the electrode parts 510 and 520 to discharge the atmosphere under a lower pressure state.

In an exemplary embodiment, the controller may control the elevating part 310 to implement relative movement of the isolation unit 400 relative to the placement unit 200.

In an exemplary embodiment, the controller may control the plasma reaction path and/or the discharge intensity of the plasma by controlling the operations of the power supply unit 530 and the electrode parts 510 and 520. Additionally, the controller may control the plasma surface treatment to occur sequentially on the object to be treated (IM). Additionally, the controller may control the light-transmissive member 915 and/or the conductive member 905 of the isolation unit 400, thereby allowing plasma treatment to be visually confirmed from the outside during a specific section or through a specific region.

In an exemplary embodiment, a plasma reaction color resulting from the reaction between the object to be treated and the plasma may be detected (1010). For example, the apparatus 10 may include one or more color detection sensors or brightness detection sensors. With such sensors, the plasma reaction color generated on the outer surface of the object to be treated (IM) during plasma reaction can be detected.

By way of example, and not limitation, a correlation between a density of ionized gas and a plasma reaction color can be induced. As the density of ionized gas increases, the plasma reaction color may have a dark purple tint. Additionally, as the density of the ionized gas decreases, the plasma reaction color may have a light purple tint. Accordingly, a partial pressure of the gas converted into plasma can be confirmed according to the plasma reaction color resulting from the plasma discharge.

In an exemplary embodiment, a correlation between the type of gas to be discharged and a plasma reaction color can be induced. That is, when the type of gas to be discharged is different, the plasma reaction color may also be different. For example, since the quantized energy value is different depending on the level of the electron orbit for each element, light of different colors may be emitted during an excitation process and/or a light emission process. More specifically, a process in which atoms are ionized may include a process in which electrons constituting discharged gas molecules are accelerated by an electric field. During such an acceleration process, electrons collide with atoms, which can cause the atoms and electrons to dissociate. When a dissociated electron enters an excited state that is higher than its original energy state, there may be force by which the electron intends to return to the original energy state. In the process in which the dissociated electron returns to its original energy state, excess energy is emitted in the form of light, and a color of the emitted light can correspond to a plasma reaction color. For example, argon may have a purple tint, nitrogen may have an orange or yellow tint, hydrogen may have a rose-colored tint, carbon dioxide may have a white tint, neon may have an orange-colored tint, mercury vapor may have a blue-green tint, and oxygen may have an orange-colored tint.

As described above, the apparatus according to an exemplary embodiment of the present disclosure may detect the plasma reaction color generated on the outer surface of the object to be treated (IM) during the plasma reaction by the sensor, and accordingly determine the type and/or partial pressure of the discharged gas. Information about the determined type and/or partial pressure of the gas may be transferred to the user through, for example, an output unit.

In an exemplary embodiment, the controller may generate information about impurities in the object to be treated (IM) based on the plasma reaction color resulting from the reaction between the object to be treated (IM) and the plasma (1020). For example, as a plasma reaction occurs on the object to be treated (IM), impurities present on the outer surface of the object to be treated (IM) react with the plasma. Accordingly, an amount and/or type of impurity may be determined in accordance with a color of the plasma reaction.

In an exemplary embodiment, the controller may generate information about performance of the plasma reaction to the object to be treated (IM) based on the plasma reaction color resulting from the reaction between the object to be treated (IM) and the plasma (1030). For example, as a plasma reaction occurs on the object to be treated (IM), qualitative information as to whether the plasma reaction occurs well, the intensity of the plasma reaction, and the like may be acquired according to the color of the plasma reaction occurring on the outer surface of the object to be treated (IM).

The information about impurities and the information about the performance may be stored while mapped with pre-stored color or brightness information. Additionally, depending on the implementation aspect, the information about the impurities and the information about the performance may also be generated by a pre-trained artificial intelligence-based module of the controller. In such a case, the artificial intelligence-based module may be pre-trained by a learning dataset including images of plasma reactions and corresponding labeling information (e.g., class information corresponding to a type of impurity, an amount of impurity, and a degree of performance).

The information generated by the controller may be transferred as a visual output and/or an auditory output through the output unit of the apparatus 10. Accordingly, the user can more easily determine whether the apparatus 10 is operating favorably, and when the performance of the plasma reaction is not high, the user can inspect the apparatus 10 or the object to be treated (IM) more easily.

In this way, the apparatus 10 according to an exemplary embodiment of the present disclosure can visually show the plasma treatment process to the user and provide the user with various types of analysis information related to the plasma treatment. Accordingly, the apparatus 10 according to an exemplary embodiment of the present disclosure can provide the user with high reliability for plasma treatment, resulting in a high user experience.

### Mode for Invention

As described above, the relevant content has been described in the best mode for implementing the invention.

### Industrial Applicability

The invention can be used for an apparatus using plasma for surface treatment of an object to be treated.

What is claimed is:

1. An apparatus for plasma surface treatment, comprising:
a placement unit on which at least one of an object to be treated, a storage container for storing the object to be treated, or a gripping device for gripping the object to be treated is placed;
an isolation unit configured to form a sealed space in which an inside thereof is isolated from an external environment during an operation period of the apparatus, the isolation unit having at least one surface comprising a light-transmissive member; and
a treatment unit configured to discharge plasma in the sealed space by forming electric field around the object during the operation period of the apparatus, and
wherein light generated by the plasma is transmitted through the light-transmissive member.

2. The apparatus of claim 1, wherein the isolation unit is configured such that the at least one surface allows transmission of visible light from the sealed space to an outside during at least one section of the operation period of the apparatus.

3. The apparatus of claim 1, wherein the isolation unit is configured to allow transmission of visible light corresponding to a wavelength range of a region that reacts with plasma in the sealed space, during at least one section of the operation period of the apparatus.

4. The apparatus of claim 3, wherein the region that reacts with the plasma corresponds to the object to be treated.

5. The apparatus of claim 1, wherein the light-transmissive member is positioned at a position where the plasma is discharged.

6. The apparatus of claim 1, wherein the isolation unit is configured to move from a first position where the isolation unit is accommodated inside an upper member defining a shape of at least a part of an upper part of the apparatus to a second position where the isolation unit forms, along with the placement unit, the sealed space, wherein at least a part of the isolation unit is accommodated in the upper member when the isolation unit is in the first position, wherein the isolation unit is taken out to outside of the upper member, while the isolation unit moves from the first position to second position, wherein one surface of the isolation unit comes into contact with one surface of the placement unit by relative movement of the isolation unit and the place unit so that an inside of the isolation unit can be formed with the sealed space, when the isolation unit is in the second position, and wherein the plasma discharged by the treatment unit occur inside of the isolation unit after forming the sealed space.

7. The apparatus of claim 6, wherein the operation period of the apparatus is divided into a plurality of sections and the sections comprise:
a first section during which the object to be treated and the placement unit are connected to each other,
a second section during which the sealed space that blocks the object to be treated (IM) from the external environment is formed by the relative movement of the isolation unit and the placement unit,
a third section during which plasma surface treatment is performed for the object to be treated by forming an electric field in the sealed space in a state in which the sealed space is formed,
a fourth section during which the object to be treated is present inside the sealed space after the plasma surface treatment is completed, and
a fifth section during which the sealed space is released by the relative movement of the isolation unit and the placement unit, the object to be treated is exposed to the external environment, and at least a part of the isolation unit is inserted into inside of the upper member.

8. The apparatus of claim 6, wherein when the isolation unit is located at the first position, the isolation unit is not visible from an outside, and when the isolation unit is located at the second position, both the isolation unit and the object to be treated are visible from the outside, and
wherein while the plasma surface treatment is performed for the object to be treated in a state in which the isolation unit is moved to the second position, a region of the object to be treated that reacts with plasma in the sealed space formed by the isolation unit changes dynamically.

9. The apparatus of claim 1, wherein the object to be treated reacts with plasma sequentially along a longitudinal direction of the sealed space.

10. The apparatus of claim 1, wherein an inner wall of the isolation unit is made of a dielectric barrier layer, and
wherein when the isolation unit and the placement unit are in contact with each other and form the sealed space, power is applied to a first electrode which is provided in the placement unit and a second electrode which is provided in the isolation unit, so that plasma discharge occurs inside of the isolation unit forming the sealed space.

11. The apparatus of claim 1, wherein a region of the object to be treated that reacts with the plasma in the sealed space is formed based on a structure of an exhaust unit connected to the sealed space, a dielectric structure inside the sealed space, and a structure of an electrode formed inside the sealed space.

12. The apparatus of claim 11, wherein the object to be treated reacts with plasma sequentially in a direction of a first electrode located proximal to the placement unit, wherein at least a part of the first electrode is exposed to the inside of the sealed space formed by the isolation unit, and wherein the first electrode is electrically connected to the object to be treated present in the sealed space, a container in which the object to be treated is stored, or at least a part of the gripping device configured to grip the object to be treated.

13. The apparatus of claim 1, wherein the isolation unit configured to form a sealed space in which an inside thereof is isolated from an external environment during an operation period of the apparatus.

14. The apparatus of claim 1, wherein at least a part of the storage container comprises a light-transmissive member so that when the object to be treated is placed on the placement unit while stored in the storage container, the object to be treated is visible from the external environment during the operation period of the apparatus.

15. The apparatus of claim 1, wherein at least a part of the gripping device comprises a light-transmissive member so that when the object to be treated is placed on the placement unit while gripped by the gripping device, the object to be treated is visible from the external environment during the operation period of the apparatus.

16. The apparatus of claim 15, wherein at least a part of the gripping device comprises a surface at a position corresponding to a position on the gripping device where the object to be treated is present.

17. The apparatus of claim 1, wherein the at least one surface of the isolation unit further comprises a conductive member, wherein the conductive member operates as at least a part of an electrode that forms the electric field inside the sealed space.

18. The apparatus of claim 1, wherein at least a part of a surface of the sealed space comprises the light-transmissive member and at least a part of a surface of the sealed space comprises a conductive member.

19. The apparatus of claim 1, wherein the isolation unit further comprises a conductive member disposed to occupy a partial region of the light-transmissive member along a region occupied by the light-transmissive member in the sealed space.

20. The apparatus of claim 1, wherein the isolation unit further comprises a conductive member and wherein at least a part of regions occupied by the conductive member and the light-transmissive member overlaps.
